(19) United States
(12) Patent Application Publication
(10) Pub. No.: US 2021/0332139 A1
(43) Pub. Date: Oct. 28, 2021

(54) MODULATION OF LEUKOCYTE ACTIVITY IN TREATMENT OF NEUROINFLAMMATORY DEGENERATIVE DISEASE

(71) Applicant: Leuvas Therapeutics

(72) Inventor: Gabriela Constantin, San Floriano (IT)

(21) Appl. No.: 17/369,670

(22) Filed: Jul. 7, 2021

Related U.S. Application Data
(62) Division of application No. 15/602,659, filed on May 23, 2017.
Application No. 15/602,659 is a continuation of application No. 14/506,391, filed on Oct. 3, 2014, now abandoned.
(60) Provisional application No. 61/886,562, filed on Oct. 3, 2013.

(51) Int. Cl. C07K 16/28

(52) U.S. Cl. CPC C07K 16/2845; C07K 16/28; A61K 2039/505; C07K 16/2836; C07K 16/2821

(57) ABSTRACT

Methods for treating and reducing the progression of neurodegenerative diseases, including, without limitation Alzheimer's disease, are provided. The methods of the invention reduce or deplete neutrophil/myeloid cells in the region of the brain by blocking neutrophil/myeloid cell adhesion and interaction with the vascular endothelium, by blocking infiltration of neutrophil/myeloid cells into the brain, by reducing motility of neutrophil/myeloid cells in the parenchyma, by blocking Aβ-induced activation and adhesion of neutrophil/myeloid cells, and/or by blocking Aβ-induced integrin activation, degranulation and/or ROS release in neutrophil/myeloid cells.

### CROSS REFERENCE

This application claims benefit and is a Divisional of application Ser. No. 15/602,659, filed May 23, 2017, which claims benefit of application Ser. No. 14/506,391 filed Oct. 3, 2014, now abandoned, which claims benefit of U.S. Provisional Patent Application No. 61/886,562, filed Oct. 3, 2013, which applications are incorporated herein by reference in their entirety.

### FIELD OF THE INVENTION

The present invention relates to the field of neurology and pharmacology. More specifically, the present invention describes a method for the prevention and treatment of Alzheimer's disease and other neurodegenerative disease. The discoveries described show that blockade of the presence, trafficking, activation, adhesion and/or function of neutrophils and/or other myeloid cells prevents and/or reduces cognitive decline, amyloid-beta deposition, tau phosphorylation, microglial activation, and normalizes pre- and post-synaptic protein levels in Alzheimer's disease and thus constitutes a therapeutic approach to treat and/or prevent Alzheimer's disease.

### BACKGROUND OF THE INVENTION

### Stages and Treatment of Alzheimer's Disease: Unmet Medical Need

Alzheimer's disease (AD) is the most common form of disabling cognitive impairment in the elderly population. The increase in life expectancy of the population and the lack of effective treatments for AD continue to lead to a rapid increase in patients with AD, which represents an untenable burden on the world population. AD is reported to be the sixth leading cause of death in the US, more than 5.2 million Americans are living with the disease, 1 in 3 senior citizens dies with AD or other dementia, AD will cost the US ˜$203 billion, and costs are expected to rise to $1.2 trillion by 2050. AD is the only cause of death among the top 10 causes in America without a way to prevent, cure or even slow its progression.

The clinical progressive decline of cognitive and behavioral symptoms is concordant with neuronal loss, synaptic dysfunction and atrophy in brain regions linked to learning and memory. Two pathophysiological hallmarks of AD are well characterized: accumulation of amyloid-beta (Aβ) peptide into amyloid plaques in the extracellular brain parenchyma and the formation of tangles inside neurons as a result of abnormal phosphorylation of the microtubule-associated protein tau. Amyloid deposits and tangles are accompanied by a marked loss of neurons in the neocortex and hippocampus.

AD is ultimately fatal. Death generally occurs within 3 to 9 years after diagnosis. There is no cure. There is no effective treatment for prevention, treatment or slowing of decline for AD patients. The U.S. Food and Drug Administration have approved five drugs that temporarily improve symptoms. The effectiveness of these drugs varies across the population, but at best they are only moderately effective in stabilizing or improving cognitive and functional symptoms for 6-12 months. None of the treatments available today alters the underlying course of this terminal disease. Clearly there is an urgent unmet medical need for effective therapeutics and approaches to treat AD.

There are 7 stages during the course of human disease. Stage 1 is characterized by normal cognitive function. Stage 2 is characterized by very mild cognitive decline and the patient may have memory lapses, but no symptoms of dementia can be detected. Stage 3 is mild cognitive decline and is considered an early-stage AD. During a detailed medical interview, doctors may be able to detect problems in memory or concentration. Common difficulties during stage 3 include: problems with the right word or name, trouble remembering names when introduced to new people, noticeable difficulty performing complex tasks, loosing or misplacing objects, increasing trouble with planning or organizing. Stage 4 is moderate cognitive decline and is equivalent of mild or early-stage Alzheimer's disease. During stage 4 the symptoms are forgetfulness of recent events, impaired ability to perform challenging mental arithmetic, greater difficulty performing complex tasks, forgetfulness about one's own personal history, becoming moody or withdrawn, especially in socially or mentally challenging situations. Stage 5 is moderately severe cognitive decline and is considered a moderate or mid-stage AD. During this stage patients present noticeable gaps in memory and thinking and start to need help with day-to-day activities: they are unable to recall their own address, telephone number, become confused about where they are or what day it is, have trouble with simple mental arithmetic. Stage 6 includes severe cognitive decline and is considered a moderately severe or mid-stage AD; memory continues to worsen in these patients, personality changes may take place and individuals need extensive help with daily activities. Stage 7 is a very severe disease and is considered the severe or late-stage AD. In this final stage of disease, individuals lose the ability to respond to their environment, to carry a conversation and, eventually, to control movement.

Neuronal inclusions comprised of the microtubule-associated protein tau are found in many neurodegenerative diseases, commonly known as tauopathies. In AD, the most prevalent tauopathy, mis-folded and/or hyperphosphorylated tau is likely a key pathological agent. Tau stabilizes microtubules within cells and is particularly abundant in neurons. Hyperphosphorylation and/or misfolding of tau results in the formation of neurofibrillary tangles inside nerve cell bodies resulting in disintegration and collapse of the neuron's transport system resulting in malfunction of neuronal function and eventually death of the neurons. Reduction or elimination of phosphorylation or misfolding of tau represents a potential strategy to treat tauopathies, including AD.

The amyloid cascade hypothesis has been very influential in the research and development of therapeutics for AD. The hypothesis posits that deposition of Aβ in the brain parenchyma initiates a sequence of events that ultimately leads to AD dementia.

Accordingly, one of the major approaches to disease modification over the past decade or two has been the targeting of Aβ plaque deposition and accumulation in the brain. Unfortunately, all of the purely Aβ-centric approaches have failed to show clinical benefit in mild to moderate patients, including two monoclonal antibodies that bind Aβ, bapineuzumab and solanezumab, as well as small molecules tramiprosate (which binds soluble Aβ) and semagacestat. These results indicate that removal of Aβ-plaque is insufficient to give clinical benefit in mild to moderate AD. Ad hoc subset analysis of the solanezumab trial suggest potential efficacy if given in early, mild disease.

Aβ is widely accepted to be a major contributor to the pathogenesis of AD; however the mechanism by which Aβ exerts neurotoxicity is poorly understood. Aβ plaques are a dominant feature in AD brain and genetic analysis of patients with early onset AD support a clear role for Aβ in disease; however, there is a no correlation between Aβ plaque load and clinical symptoms and there is a spatial and temporal disconnect between the levels and location of Aβ in the brain and neuronal loss. Thus, the deleterious effects of Aβ in AD appear to be mediated by a mechanism soluble factors and/or motile cells.

Aβ targeted interventions may yield clinical benefit if they are initiated very early, before secondary mechanisms, severe synaptic dysfunction, irreversible widespread cell loss, and neurodegeneration have occurred. Thus, reduction in Aβ levels during early disease remains an important goal in the treatment of AD; however, it is likely that effective therapy will require parallel, combination approaches for preventing and mitigating neurodegeneration. The combination of removal or reduction of the inflammatory trigger and blunting of the inflammatory response to the trigger may provide a superior approach.

### Role of Inflammation

Epidemiological studies have shown that use of non-steroidal anti-inflammatory drugs (NSAIDs) is correlated with reducing the risk for AD, suggesting that neuroinflammation might play a role in early phase of disease; however, long-term, placebo-controlled clinical trials with both non-selective and cyclooxygenase-2 selective NSAIDS have shown that NSAIDS did not improve cognitive function in AD.

Analysis of microarray-gene expression studies in people with AD and those with mild cognitive changes at increased risk of developing AD compared to normal controls show that a number of genes encoding cell adhesion molecules, including L-, P-, and E-selectin, PSGL1, ICAMs, VCAM, MadCam, CDII/CD18 and alpha 4 were modestly upregulated, along with many other immune-related genes. Further they found increased numbers of basophils in people with mild cognitive impairment (MCI) and AD, and increased monocytes in people with AD diagnosis.

Overall, these authors suggest the presence of a chronic peripheral low-grade innate immune response in people with MCI and AD (Lunnon et al, 2012, J Alzheimers Dis.; 30(3):685-710). Numbers of circulating neutrophils and their intracellular signaling are altered in aged AD patients relative to age-matched or control young subjects (Song et al, 1999, Psychiatry Res. 85(1):71-80). The ratio of neutrophils to lymphocytes (NLR) can be thought of as an indicator of the body's inflammatory status and has been used to predict prognosis in miscellaneous diseases including congestive heart disease and malignancy. The mean NLR has been shown to be slightly higher in AD than in patients with normal cognitive function (3.21+/−1.35 vs. 207+/−0.74, p<0.001; Kuyumcu et al., 2012, Dement Geriatr Cogn Disord. 34(2):69-74).

Studies describing changes in immunologic parameters in moderately severe AD patients, such as an increase in the HLA-DR and CD4 markers and a slight decrease in CD8+ subset of T cells, support the hypothesis of a peripheral T-cell immune reaction in AD, which may be correlated with the clinical stage of the disease (Shalit et al., 1995, Clin Immunol Immunopathol.; 75(3):246-50.) Peripheral blood neutrophils from patients with AD showed a slight increase in basal expression of CD11b (32.9+/−2.6 relative fluorescence units; RFU) compared to control subjects (21.1+/−1.6 RFU); however, stimulation of the neutrophils with fMLP resulted in a huge increase in CD11b expression in both groups, to 120.3 and 115.2 RFU for AD and control patients, respectively, with no significant differences in activation status between groups. There was a correlation between RFU and disease state in the sporadic AD patients (Scali C et al., 2002, Neurobiol Aging; 23(4):523-30). The conclusion was that inflammatory and immune-related reactions in the peripheral blood cells reflect the severity of changes occurring in the AD brain. The elevated basal level of CD11 b on neutrophils suggested a state of neutrophil “alert” activation, which was attributed to increased TNF, IL-6 and ICAM as well as other inflammatory-immune markers in the serum of AD patients. These elevated levels were in part considered to be markers of disease, potentially useful in monitoring the progression of AD, and not involved in disease, since post-mortem brains of AD revealed no neutrophil invasion (Scali et al., 2002, Neurobiol Aging; 23(4):523-30).

Cathepsin G-containing cells, identified as neutrophils based on morphology, were found in the parenchyma and inside vessels in AD as well as age-matched normal brain (Savage 1994). Cathepsin G, a protease that is capable of cleaving partially purified beta-amyloid precursor protein produced in baculovirus (Savage M J et al., 1994, Neuroscience. June; 60(3):607-19). is expressed by neutrophils as well as microglia and other cells. The Cathepsin G positive cells were also found in equivalent numbers in age-matched normal brains and in AD brains they were clearly not localized to amyloid deposits. These authors suggest that a circulating source of Aβ may be generated by cathepsin G from enzyme released from neutrophils acting on APP substrate from platelets, endothelial cells and/or lymphocytes, and raise the possibility that cathepsin G could be involved in APP processing locally within the brain parenchyma; however conclude that since there was no association of neutrophils with amyloid deposition and no generalized increase in the number of neutrophils in AD brain, a primary role for neutrophils and cathepsin G in amyloid deposition within brain parenchyma is unlikely (Savage M J et al., 1994, Neuroscience. June; 60(3):607-19).

Based on this type of evidence, inflammation has been suggested as a possible driving force, simple bystander response, or potentially a beneficial response in AD (Wyss-Coray, T, 2006, Nature Medicine; 12(9):1005). Mild systemic inflammation is associated with disease, with peripheral neutrophils and/or myeloid and other leukocytes existing in a semi-activated state; however, direct or indirect effects of neutrophil/myeloid interaction with the endothelium and/or myeloid/neutrophil invasion into the brain have not been associated with the disease.

Microglia and astrocytes are the cells most often cited as the key cells involved in this inflammatory process and there is ample evidence showing that these cells are dysfunctional in AD brain as well as in transgenic animal models. Microglia are resident immune cells in the brain and are exquisitely sensitive to disturbances of brain homeostasis as well as to systemic events. Microglia have been shown to become activated in a progressive and age-dependent manner and activation is correlated with the onset of fibrillar and Aβ plaque accumulation and tau hyper-phosphorylation. Activated microglia in brains of AD patients are distributed with both Aβ plaques and neurofibrillary tangles and are involved in excessive tau phosphorylation that is related to tangle development in AD. Further, when microglia interact with the deposited fibrillar forms of beta-amyloid it leads to activation of the microglia cells and results in the synthesis and secretion of cytokines and other neurotoxic proteins.

Aβ42 can be directly neurotoxic or can activate mononuclear phagocytes to secrete neurotoxins. It has been shown to have chemokine-like properties for microglia and the search for receptors has yielded several candidate molecules including the scavenger receptor, CD36 and the receptor for advanced glycation end products (RAGE). Aβ42 has been shown to act through FPR in neuronal cells (Lorton D et al., 2000, Neurobiol Aging. 2000 May-June; 21(3):463-73) and FPRL1/FPR2 in neuronal cells. The neutrophil formyl peptide receptors (FPR) FPR1 and FPR2 are members of the C-protein coupled receptor family. Both pro- and anti-inflammatory signals can be generated by occupation of FPR's. These receptors have been come a therapeutic target for the development of novel drugs to reduce injuries in inflammatory diseases including RA, cardiovascular disease, asthma and more. It has been suggested that targeting inflammatory glia cytokine pathways can suppress Aβ-induced glial-mediated neuroinflammation.

A need exists for new approaches and new therapeutics to treat AD. The present invention addresses this need, and provides surprising benefits of blocking neutrophil and/or myeloid cell activation, adhesion and/or invasion of the brain to treat Alzheimer's and other neurodegenerative disease.

### BRIEF SUMMARY OF THE INVENTION

Compositions and methods are provided for the prevention and treatment of neurodegenerative disease in an individual, including without limitation Alzheimer's disease (AD). In the methods of the invention, an individual suffering from, or pre-disposed to, a neurodegenerative disease is contacted with an effective dose of an agent that reduces the presence or activity of myeloid cells and/or neutrophils in the region of the brain, which region may include the vasculature of the brain. The agent is provided for a period of time sufficient to reduce the presence or activity of myeloid cells and/or neutrophils in the brain and/or vasculature thereof; which may include a reduction of interactions with the brain vasculature and/or at the site of neurodegenerative lesions, e.g. at plaques associated with AD.

Although the presence or activity of myeloid cells and/or neutrophils within the brain, including the vasculature of the brain, is reduced, in some embodiments the inhibitor is not required to cross the blood brain barrier, and may be administered systemically.

Agents of interest for use in the methods of the invention modulate or interfere with at least one pathway of myeloid cell and/or neutrophil trafficking or activation. Depending on the specific pathway, an agent may be an inhibitor, blocking or depleting agent, e.g. an antibody that depletes a targeted cell population, an agonist, or an antagonist of one or more of these pathways. For example an agent may block the activity of a protein that acts in one or more of these pathways, e.g. a chemoattractant, a protein tyrosine kinase, an adhesion molecule, etc.

Pathways of interest for intervention by the methods of the invention include (i) depletion of neutrophil/myeloid cell populations systemically or locally in the brain; (ii) blocking neutrophils/myeloid cell adhesion and crawling; (iii) blocking transmigration and infiltration of neutrophils/myeloid cells into the brain; (iv) blocking cell-cell interactions between neutrophil/myeloid cells and endothelial cells and/or neural cells; (v) blocking neutrophil/myeloid cell extracellular-matrix interactions; (vi) reducing motility of neutrophils/myeloid cells in the brain parenchyma; (vii) blocking Aβ-induced activation and adhesion of neutrophils/myeloid cells; (viii) blocking intracellular signaling controlling adhesion and activation; (ix) blocking neutrophil activation and/or degranulation; (x) blocking release of reactive oxygen species, proteases, cytokines, lipid mediators or other damaging agents from myeloid cells and/or neutrophils; (xi) blocking neutrophil/myeloid cell activation leading to increased affinity and valency resulting from clustering of integrin receptors that increases binding; (xii) blocking formation of neutrophil extracellular traps (NETS) in brain vessels or parenchyma; (xiii) blocking neurodegenerative processes including synaptic dysfunction and/or degradation; (xiv) reducing activation and/or number of microglial cells.

In one embodiment of the invention, an effective dose or dosing regimen of an agent that targets adhesion molecules involved in leukocyte trafficking or extravasation, including but not limited to: integrins and their ligands, e.g. ICAM-1, LFA-1, CD11a, CD11b, CD11c, CD18, alpha-4 integrins and their ligands VCAM-1 and MAdCAM-1, etc.; CD49; E-, P- and L-selectin and their ligands, e.g. including but not limited to PSGL-1, CD44, CD43, hyaluronan, glycolipids, etc.; is administered to treat or prevent AD or other neurodegenerative disease. In some embodiments, the agent inhibits the interaction between an adhesion molecule involved in leukocyte trafficking to the brain, and its ligand.

In one embodiment of the invention, an effective dose or dosing regimen of an agent targeting protein tyrosine kinases, including but not limited to, Syk, Abl, JAK3, Jak2, and BTK and MAPK; and/or PI3K, is administered to treat or prevent AD or other neurodegenerative disease.

In one embodiment of the invention provides for administration of an agent targeting fPR on neutrophils/myeloid cells. In this embodiment the agent is not required to cross the BBB.

In some embodiments the individual is diagnosed with AD or other neuroinflammatory disease prior to treatment, which diagnosis may include, without limitation, analysis of one or a combination signature molecular networks shown in the art to be dysregulated in AD or other neuroinflammatory disease.

In some embodiments the efficacy of treatment, e.g. dosing and/or dosing regimen, is tracking by monitoring one or more biomarkers selected from (i) the number of circulating neutrophils/myeloid cells; (ii) the number of brain-resident neutrophils/myeloid cells; (iii) activation status of circulating neutrophils/myeloid cells; (iv) activation status of brain-resident neutrophils/myeloid cells; (v) adhesion capability of circulating neutrophils/myeloid cells; (vi) adhesion capability of brain-resident neutrophils/myeloid cells; (vii) inflammatory markers in blood; (viii) inflammatory markers in cerebrospinal fluid; (viii) neurodegenerative markers in CSF or blood. In such methods, monitoring may be performed prior to treatment, and/or following treatment, and may be performed at intervals during the course of treatment.

The present invention is based, in part, on the discoveries that neutrophils/myeloid cells are present in Alzheimer's disease (AD) brains and are largely associated with areas of disease, but are not present in normal brain; that soluble and fibrillary amyloid beta can trigger integrin activation in neutrophils, causing rapid adhesion that vascular adhesion molecules are expressed on vascular endothelial cells in AD brain during early disease; and that short-term therapeutic blockade of neutrophil activation, adhesion and/or invasion in to brain prevents cognitive decline, Aβ deposition and tau phosphorylation in models of AD; and that short term therapeutic treatment at early to mid-stage disease in mouse models of AD to prevent neutrophil/myeloid cell activation/adhesion, neutrophil/myeloid-endothelial cell interaction, neutrophil/myeloid cell invasion into the brain and/or neutrophil/myeloid neural cell interaction resulted in short and long-term blockade of cognitive decline and preservation of cognitive function. In some embodiments the therapeutic agent does not cross the blood brain barrier.

### DETAILED DESCRIPTION OF THE EMBODIMENTS

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The methods of the invention find use in a wide variety of animal species, particularly including mammalian species. Animal models, particularly small mammals, e.g. murine, lagomorpha, etc. are of interest for experimental investigations. Other animal species may benefit from improvements in in vitro fertilization, e.g. horses, cattle, rare zoo animals such as panda bears, large cats, etc. Humans are of particular interest. Individuals of interest for treatment with the methods of the invention include, without limitation, those suffering from Alzheimer's disease.

### Definitions

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms.

A “therapeutically effective amount” or “efficacious amount” means the amount of a compound that, when administered to a mammal or other subject for treating a disease, is sufficient to effect such treatment for the disease. The “therapeutically effective amount” will vary depending on the compound, the disease and its severity and the age, weight, etc., of the subject to be treated.

Alzheimer's disease. Alzheimer's disease is a progressive, inexorable loss of cognitive function associated with an excessive number of senile plaques in the cerebral cortex and subcortical gray matter, which also contains β-amyloid and neurofibrillary tangles consisting of tau protein. The common form affects persons >60 yr old, and its incidence increases as age advances. It accounts for more than 65% of the dementias in the elderly.

The cause of Alzheimer's disease is not known. The disease runs in families in about 15 to 20% of cases. The remaining, so-called sporadic cases have some genetic determinants. The disease has an autosomal dominant genetic pattern in most early-onset and some late-onset cases but a variable late-life penetrance. Environmental factors are the focus of active investigation.

In the course of the disease, neurons are lost within the cerebral cortex, hippocampus, and subcortical structures (including selective cell loss in the nucleus basalis of Meynert), locus caeruleus, and nucleus raphae dorsalis. Cerebral glucose use and perfusion is reduced in some areas of the brain (parietal lobe and temporal cortices in early-stage disease, prefrontal cortex in late-stage disease). Neuritic or senile plaques (composed of neurites, astrocytes, and glial cells around an amyloid core) and neurofibrillary tangles (composed of paired helical filaments) play a role in the pathogenesis of Alzheimer's disease. Senile plaques and neurofibrillary tangles occur with normal aging, but they are much more prevalent in persons with Alzheimer's disease.

Neurons communicate with one another at specialized contact sites called synapses composed of pre- and post-synaptic compartments. Synapse loss and defects in the presynaptic compartment and overall synaptic dysfunction are considered a significant factor contributing to memory loss in Alzheimer's disease, correlates better with cognitive dysfunction than amyloid deposition and tangle formation, and is recognized to be a primary pathological target for treatment. Synaptotagmin (or synaptotagmin 1, encoded by gene SYT1) is a pre-synaptic protein that is reduced in brain and CSF of patients with AD, including patients at an early stage of disease, as compared with age-matched healthy controls and has been suggested as a biomarker for synaptic pathology in Alzheimer disease. PSD-95 is a postsynaptic protein that is also reduced at later time points as the pathologies advance.

The essential features of dementia are impairment of short-term memory and long-term memory, abstract thinking, and judgment; other disturbances of higher cortical function; and personality change. Progression of cognitive impairment confirms the diagnosis, and patients with Alzheimer's disease do not improve.

AD animal models. In 2011 the National Institute on Aging and Alzheimer's Association (NIA-AA) proposed a new framework for characterizing preclinical AD in man. Stage 1 includes abnormal levels of Aβ; stage 2 includes abnormal levels of Aβ and evidence of brain injury; stage 3 abnormal levels of Aβ and evidence of brain injury plus subtle cognitive changes. The clinical definitions of stages 1-6 are described above.

The 5XFAD mouse model shows abnormal Aβ deposition, memory deficits by 4 months of age and the 3xTg-AD model shows Aβ abnormalities between 3-4 months and synaptic dysfunction and learning and memory deficiency by 6 months.

Based on the new definition from the NIA-AA and the clinical stages described in the introduction/background, and the time-course of disease in the mouse models, the timing of the therapeutic interventions described in the mouse models are defined as early clinical stage 3 to 4 and preclinical stage 4 and are defined as early to mid-stage of AD.

It has been shown that patients with familial AD (FAD) present mutations of genes encoding APP itself or protease subunits such as presenilin (PS) 1 and 2 involved in APP cleavage to generate Aβ. The discovery of mutated APP and PS was the basis for generation of transgenic animal models harboring these human mutations and thus closely replicating cardinal features of AD. Transgenic mouse models have greatly advanced the understanding of AD pathogenesis. Transgenic mice overproducing human APP containing familial AD mutations show increased production of Aβ, which accumulates with age into diffuse or compact amyloid plaques. The mice show synaptic transmission deficits that often precede the formation of the plaques. Overexpression of presenilin1 further increases Aβ production and accelerates pathology. Mice overexpressing human tau protein mutants that are associated with familial forms of frontotemporal dementia and Parkinsonism linked to chromosome 17—a dementia characterized by extensive tangle formation develop neurofibrillary tangles similar to those observed in AD. Mice expressing the P301 mutant tau mimic features of human tauopathies.

A mouse model has been described, the 3xTG model, that harbors all three mutant genes, tauP^{301L}, APP^{K670N, M671L} and PS1^{M146V}. 3xTg-AD mice produces amyloid plaques and tangles, shows synaptic transmission defects, and develop age-related and progressive neuropathological phenotype in the hippocampus amygdala and cerebral cortex, the most pronounced brains structures impacted by AD pathology. Intracellular Aβ is apparent between 3 and 4 months of age in the neocortex and by 6 months in the hippocampus. Neurofibrillary alterations tau pathology (hyperphosphorylated and/or conformationally altered tau) is observed between 12 and 15 months of age. Tau-reactive dystrophic neuritis is evident in older 3xTg-AD brains (18 mo). Of note, Aβ and tau pathology initiate in different brain regions in 3xTg-AD mice (i.e. cortex for Aβ and hippocampus for tau). This is not inconsistent with the notion that Aβ influences tau pathology, but suggests that a soluble intracellular Aβ, other soluble factors and/or motile cells are involved in Aβ-mediated tau pathology. By 6 months (the earliest time points tested after baseline measurements at 1 month) synaptic dysfunction and long-term potentiation (LTP), which is involved in learning and memory) was severely impaired in 3xTg-AD mice compared to wild-type-aged matched mice.

The 5XFAD mouse model of amyloid deposition expresses five familial AD (FAD) mutations that are additive in driving Aβ overproduction. These mice overexpress both mutant human APP (695) and the Swedish (K670N, M671L), Florida (I716V), and London (V7171) Familial Alzheimer's Disease (FAD) mutations and human PS1 harboring two FAD mutations, M146L and L286V. 5XFAD mice exhibit intraneuronal Aβ accumulation by 1.5 months, amyloid deposition by 2 months, memory deficits by 4 months of age, and caspase-3 activation and neuron loss by starting at approximately 6 months old.

Two photon microscopy (TPM) has exceptional depth penetration and intrinsic optical sectioning properties allowing for high-resolution in vivo imaging. In recent years, the advent of TPM and the generation of transgenic animals which express fluorescent proteins driven by tissue-specific promoters and have allowed the direct observation of cells and their behaviors under both physiological and pathological conditions in vivo. Recent data documented and characterized some of the molecular mechanisms controlling intravascular and intra-tissue behavior of neutrophils by using TPM.

Neutrophil/myeloid cells. The discoveries outlined herein are focused largely on mouse and human neutrophils; however, in some mouse studies an anti-neutrophil antibody, RB6-8C5 was used, both to define cells in flow cytometry and for blocking/depletion experiments. RB6-8C5+ cells are largely neutrophils but also include a small population of Gr1+/Ly6C+ monocytes, thought to be a precursor of inflammatory tissue macrophages, and several other populations of myeloid cells. Neutrophil/myeloid cells may be defined herein as neutrophils plus the Gr1+/Ly6c+ monocytes.

Leukocyte trafficking and endothelial cell interactions. The endothelial interface/endothelium produces a large number of soluble factors that can influence systemic and local tissue function. Though never implicated previously in AD, interaction of neutrophils with the endothelium in other inflammatory conditions is known to result in neutrophil-mediated endothelial damage, including preeclampsia, reperfusion injury, adult respiratory distress syndrome and multiple organ failure. Once bound to the endothelium or infiltrated into the tissue, neutrophils can damage tissue through release of ROS, and proteases and drive inflammation via secretion of cytokines, chemokines, and leukotrienes.

Leukocyte recruitment is the primu movens of any immune response and is critical to the onset of inflammatory and autoimmune disease. The molecular mechanisms involved in neutrophil-endothelial cell adhesive interactions have been extensively reviewed (Ley K et al., 2007, Nat Rev Immunol. 7:678-689 and references therein). Briefly, sequential initial steps of neutrophil-endothelial interactions are tethering, rolling, activation and firm adhesion (arrest). Neutrophil tethering and rolling are mediated by selectins; L-selectin is expressed constitutively on neutrophils, whereas activated endothelial cells express E- and P-selectins. The selectins interact with their counter-receptors on leukocytes and endothelial cells.

Recruitment of neutrophils requires CD11/CD18 complexes under most circumstances; however, non-CD11/CD18-mediated neutrophil emigration has been demonstrated certain pathological conditions in CD18 deficient mice. Neutrophils are generally assumed not to express alpha-4 integrin; however, neutrophils have been shown to express alpha 4 integrin under certain conditions in vitro and in vivo and the alpha4-integrin-VCAM-1 pathway for neutrophil recruitment has been demonstrated in human disease. Alpha-4 expressing neutrophils have been identified in the circulation of septic patients, and the alpha-4 integrin pathway can be induced in neutrophils upon exposure septic plasma. The alpha-4 pathway can mediate tethering, rolling and adhesion under flow conditions on VCAM-1 and MAdCAM-1. Further, alpha-4 integrin has been shown to mediate neutrophil-induced free radical injury to cardiac myocytes, and plays a role in neutrophil migration through the extracellular matrix and connective tissue. Thus, it appears that under certain pathophysiological conditions a sufficient proinflammatory milieu can induce alpha-4 integrin-VCAM-1/MadCam-1-mediated neutrophil-endothelial interaction, adhesion, recruitment, diapedesis and migration within the tissue. The alpha-4 adhesion pathway provides therapeutic target to reduce inappropriate neutrophil adhesion without altering the normal yet critical beta-2 integrin mediated adhesive function of neutrophils.

In vitro and in vivo studies have established that leukocyte arrest is rapidly triggered by chemokines or other chemoattractants and is mediated by the binding of leukocyte integrins to immunoglobulin superfamily members, such as ICAM1 and VCAM1, expressed by endothelial cells. LFA-1 integrin is one of the most relevant to leukocyte arrest and classical chemoattractants are the most powerful physiological activators of LFA-1-mediated adhesion in vivo. Ligation of specific heterotrimeric GPCRs by chemokines activate integrins by triggering a complex intracellular signaling network within milliseconds leading to the increase of both integrin affinity and valency. Inside-out signaling induces integrins to undergo a dramatic transition from a bent low-affinity conformation to extended intermediate- and high-affinity conformations, which leads to opening of the ligand-binding pocket.

In this context, integrin activation is a key step since it mediates rolling (in certain conditions), arrest and diapedesis of activated leukocytes. The most potent agonists for integrin triggering are chemotactic factors, such as formylated peptides or chemokines. Chemotactic factors receptors trigger a very complex intracellular signaling network leading to various kinetic aspects of integrin-mediated adhesion. Overall, at least 65 signaling proteins are involved in the regulation of integrin-mediated adhesion by chemoattractants.

The selectins mediate adhesion of hematopoietic cells, including neutrophils and myeloid cells, to vascular cells and to each other. These interactions are key for host defense, immune cell surveillance and inflammation. Reversible interactions with E- and P-selectin expressed on endothelial cells mediate tethering and rolling in inflamed vascular beds. The selectins are calcium-dependent lectins that bind to glycan determinants on a variety of proteins and lipids. There are a large number of selectin ligands, including PSGL-1, CD44, E-selectin ligand, and CD43 that can play a role in myeloid cell/neutrophil interaction with the vasculature. Some of these ligands can mediate signaling cascades, for example PSGL-1 and CD44 induce signals that activate beta-2 integrin LFA-1 and ESL-1 can activate the Beta2 integrin MAC-1 in neutrophils.

ICAM-1 is expressed at basal levels in normal mouse and increased expression is seen in AD brain. The treatment of cerebral endothelial cells with oligomeric Aβ in vitro results in the expression of P-selectin. A recent study has shown increased VCAM-1 expression in microarrays from brain homogenates of the 3xTgAD model of AD in mice. Increased levels of soluble E-selectin, VCAM-1 and ICAM-1 have been found in the blood of AD patients. However, the expression of VCAM-1 and E- and P-selectin on endothelial cells in animal models of AD or human patients with AD have not been demonstrated.

PSGL-1 is a major selectin ligand on leukocytes. PSGL-1 is the predominant ligand for P-selectin, but it can also bind to E- and L-selectin under flow conditions and mediates leukocyte tethering and rolling, and can transduce signals into rolling leukocytes and into leukocytes decorated with platelets. P-selectin and PSGL-1 are key targets for inhibition of myeloid cells/neutrophil interaction with the vascular endothelium for the prevention and/or treatment of Alzheimer's disease.

CD44 is a class one transmembrane glycoprotein expressed on most vertebrate cells, including monocytes, neutrophils, lymphocytes and endothelial cells, and is involved in many cellular processes. CD44 is encoded by a single gene and has more than 40 isoforms. The heterogeneity results from posttranslational modifications such as sulfation and glycosylation as well as alternative splicing. There are at least three ligands, hyaluronate, collagen types I and VI and MAdCAM. One form is known to bind to L- and E-selectin in vitro (termed the hematopoietic cell E-/L-selectin ligand). The 80-90 Kd form is known to be expressed in brain, particularly in the white matter. In addition to diffuse labeling in white matter, CD44 is expressed by some astrocytes. Hyaluronan (a key ligand for CD44) levels significantly increase with age accompanied by an increase in CD44 in rhesus macaques. An increase in CD44 gene expression is found in lymphocytes derived from AD patients. CD44 undergoes an intramembranous cleavage to liberate its intracellular domain via a presenilin-dependent gamma-secretase activity resulting in release and secretion of an Aβ-like peptide. CD44 can also bind to hyaluronan, an extracellular matrix glycosaminoglycan. Under inflammatory conditions CD44 on endothelial cells presents hyaluronan to leukocytes and mediates rolling under flow conditions, leading to arrest and migration.

CD43, also known as leukosialin, may have pro and anti-adhesive roles. It can serve as an E-selectin ligand on inflammatory T cells and may play a role in delayed type hypersensitivity in skin inflammation. Like PSGL-1, CD43 localizes to microvilli, suggesting a role in cell tethering and may play a role in T cell and neutrophil recruitment (and has been shown to contribute to neutrophil rolling on E-selectin together with PSGL-1. It can have either a pro-adhesive or anti-adhesive role.

Galectin-3, a member of the galectin family of carbohydrate binding proteins, is widely expressed in immune cells and is thought to play a role in adhesion. Galectin-3 exists as a monomer and it has been proposed that oligomerization may control its immunomodulatory role. Galectin-3 promotes adhesion of human neutrophils to laminin and fibronectin and may play a role in the migration of neutrophils through basement membrane at inflammation sites.

Leukocyte Depletion. The depletion of a leukocyte population has been shown to be a viable approach for therapy. For example, Rituximab has been approved for the treatment of lymphoma, leukemia, transplant rejection and autoimmune disorders. This therapeutic antibody binds to CD20, which is widely expressed on B cells from the early pre-B cell stage and on to later differentiated B cells and causes the apoptosis of CD20+B cells, thus eliminating all B cells from the circulation.

Leukocyte Activation. Tyrosine kinases (PTK) are of interest for targeting leukocyte activation. PTKs are normally upstream transducers in signaling cascades, thus they may behave as massive amplifiers and regulators of signaling events. PTKs are ideal targets of pharmacological modulation in immune-related diseases, where leukocyte recruitment plays a critical role.

N-formyl peptides induce neutrophil activation and functional activation through the seven-transmembrane G protein-coupled receptor FPR1, however, neutrophils express a vast repertoire of pattern recognition receptors (PPRs), including all members of the Toll-like receptor family (with the exception of TLR2) and the C-type lectin receptors dectin 1 and CLEC2, and cytoplasmic sensors of ribonucleic acids (RIG-1 and MDA5). In addition, neutrophils express nucleotide-binding oligomerization domain protein 1 (NOD1). The sensing of pathogens and tissue damage through these PRRs, together with other cell-derived signals, activates the effector function of neutrophils, and provides a useful point of intervention for the methods of the invention.

Neutrophils are also activated by G-CSF and GM-CSF, TNF, Type I and Type II interferons and in response express a broad repertoire of cytokines, including CXC-chemokines (CXCL1, CXCL2, CXCL3, CXCL4, CXCL5, CXCL6, CXCL8, CXCL9, CSCL10 and CXCL11), CC-chemokines (CCL2, CCL3, CCL4, CCL17, CCL18, CCL19, CCL20, CCL22), pro-inflammatory cytokines (IL-1α, IL-1β, IL6, IL-7, IL-9, IL-16, IL-17A, IL-17F, IL-18 and MIF), anti-inflammatory cytokines (IL-1RA, IL-4, IL-10, TGFβ1, TGFβ2), immune-regulatory cytokines (IFNα, IFNγ, IL-12, IL-23), colony-stimulating factors (G-CSF, M-CSF, GM-CSF, IL-3 and SCF), angiogenic and fibrogenic factors (HB-EGF, HGF, FGF2, TGFα, VEGF, prokineticin2), TNF superfamily members (APRIL, BAFF, CD30L, LIGHT, LTβ, RANKL, TNF and TRAIL), and other cytokines (eg amphiregulin, BDNF, midkine, NGF, NT4, oncostatinM, PBEF). Cytokine production and release is regulated by mechanisms that act at different levels, including mRNA transcription, stability or translation and protein secretion. Some cytokines are not directly released following synthesis but are stored in intracellular pools and are secreted only when neutrophils are secreted by secretagogue-like molecules. Blocking or inhibition of cytokines provides a point of intervention for the methods of the invention.

Beyond their direct role in orchestrating the innate immune system to protect against invading pathogens, neutrophils also release an important set of proinflammatory biological modulators that mediate recruitment of additional cells to sites of infection and inflammation and amplify the innate protective response, such as reactive oxygen intermediates and cytokines, neutrophils can release a highly charged network of DNA and nuclear proteins named neutrophil extracellular traps (NETs). These NETs entrap pathogens, but also contain neutrophil secretory granule-derived serine proteases, neutrophil elastase and cathepsin G, which can regulate neutrophils and platelets and may regulate local inflammatory conditions. NETs also contain some neutrophil-derived pattern recognition (PMRs) molecules with antibody-like properties. These PMRs, including for example ficolins and peptidoglycan recognition protein short, not only enhance phagocytosis and activate complement, but also regulate inflammation. Thus, although key in the protection of the host against invading pathogens, NETs may have consequences for the host. Blocking NET activity or formation provides a useful point of intervention for the methods of the invention.

Protein kinase C (PKC) serine-threonine kinases are master regulators of proinflammatory signaling hubs, making them attractive therapeutic targets. PKC isoforms have been implicated in inflammatory processes.

When neutrophils are activated, the nicotinamide adenine dinucleotide phosphate (NADPH) oxidase is rapidly assembled to generate superoxide anions (O₂⁻) and additional reactive oxygen species (ROS). Neutrophil ROS is vital to host defense, however, release of ROS and other cytotoxic substances and subsequent by-stander tissue injury is a key mechanism common to several diseases.

Neutrophils can play a role in active induction of resolution of inflammation through production of lipid mediators. Families of endogenous anti-inflammatory and proresolving autacoid mediators have been recently discovered, including lipoxins (LXs), resolving, protectins, and maresins, which are potent regulators of neutrophil activation. Additional regulatory molecules, such as excitotoxins including as N-methyl-D-aspartate (NMDA) or kainic acid, or the NMDA receptor agonist quinolinic acid, have potent neurotoxic effects. Quinolinic acid, which is produced by activated microglia and macrophages, may be involved in neurodegenerative processes in the brain as well as in many psychiatric disorders. Injection of these types of substances into rat striatum or hippocampus results in infiltration of neutrophils into the injected areas.

Lipoxins are potent anti-inflammatory mediators for neutrophils. For example, on neutrophil activation, polyisoprenyl diphosphatate phosphatase 1 (PDP1) rapidly converts presqualene diphosphate (PSDP) to presqualene monophosphate to facilitate cell activation. Remodeling of PSDP by PDI1 that is activated by phosphor-PKCpII can be blocked by proresolving agonists, such as LXs, providing therapeutic targets to dampen neutrophil activity in disease. Neutrophils also can switch their eicosanoid biosynthesis of resolvins, such as resolvin E1, resolvin E2, resolvin D1 and resolvin D2, and protectin D1, which are derived from omega-3 essential polyunsaturated fatty acids. These pro-resolving lipid mediators and the recently described maresin 1 inhibit neutrophil transmigration and tissue infiltration. Pro-resolving lipid mediators increase expression of CCR5, which can then act as a functional decoy and scavenge for inflammatory CC-chemokines.

As used herein, an “antagonist,” refers to a molecule which, when interacting with (e.g., binding to) a target protein, decreases the amount or the duration of the effect of the biological activity of the target protein (e.g., interaction between leukocyte and endothelial cell in recruitment and trafficking). Antagonists may include proteins, nucleic acids, carbohydrates, antibodies, or any other molecules that decrease the effect of a protein. Unless otherwise specified, the term “antagonist” can be used interchangeably with “inhibitor” or “blocker”.

The term “agent” as used herein includes any substance, molecule, element, compound, entity, or a combination thereof. It includes, but is not limited to, e.g., protein, oligopeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a molecule of interest but which has been modified in a targeted and controlled manner, by replacing a specific substituent of the reference molecule with an alternate substituent. Compared to the starting molecule, an analog may exhibit the same, similar, or improved utility. Synthesis and screening of analogs, to identify variants of known compounds having improved traits (such as higher potency at a specific receptor type, or higher selectivity at a targeted receptor type and lower activity levels at other receptor types) is an approach that is well known in pharmaceutical chemistry.

Antagonists of interest include antibodies specific for one or more adhesion molecules involved in leukocyte recruitment or trafficking to the central nervous system. Also included are soluble receptors, conjugates of receptors and Fc regions, and the like. Generally, as the term is utilized in the specification, “antibody” or “antibody moiety” is intended to include any polypeptide chain-containing molecular structure that has a specific shape which fits to and recognizes an epitope, where one or more non-covalent binding interactions stabilize the complex between the molecular structure and the epitope. The archetypal antibody molecule is the immunoglobulin, and all types of immunoglobulins (IgG, IgM, IgA, IgE, IgD, etc.), from all sources (e.g., human, rodent, rabbit, cow, sheep, pig, dog, other mammal, chicken, turkey, emu, other avians, etc.) are considered to be “antibodies.” Antibodies utilized in the present invention may be polyclonal antibodies, although monoclonal antibodies are preferred because they may be reproduced by cell culture or recombinantly, and may be modified to reduce their antigenicity.

Antibody fusion proteins may include one or more constant region domains, e.g. a soluble receptor-immunoglobulin chimera, refers to a chimeric molecule that combines a portion of the soluble adhesion molecule counterreceptor with an immunoglobulin sequence.

The immunoglobulin sequence preferably, but not necessarily, is an immunoglobulin constant domain. The immunoglobulin moiety may be obtained from IgG1, IgG2, IgG3 or IgG4 subtypes, IgA, IgE, IgD or IgM, but preferably IgG1 or IgG3.

In addition to entire immunoglobulins (or their recombinant counterparts), immunoglobulin fragments comprising the epitope binding site (e.g., Fab′, F(ab′).sub.2, or other fragments) are useful as antibody moieties in the present invention. Such antibody fragments may be generated from whole immunoglobulins by ficin, pepsin, papain, or other protease cleavage. “Fragment” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

Small molecule agents encompass numerous chemical classes, though typically they are organic molecules, e.g. small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of GABA.sub.A inhibitors can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25, 1994; Burbaum et al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

Candidate antagonists can be tested for activity by any suitable standard means. As a first screen, the antibodies may be tested for, binding against the activation molecule, adhesion molecule, etc. As a second screen, candidates may be tested for binding to an appropriate cell line, e.g. leukocytes or endothelial cells, or to primary tissue samples. For these screens, the candidate may be labeled for detection (e.g., with fluorescein or another fluorescent moiety, or with an enzyme such as horseradish peroxidase). After selective binding to the target is established, the candidate produced as described below, may be tested for appropriate activity, including the ability to block leukocyte recruitment to the central nervous system in an in vivo model, such as an appropriate mouse or rat epilepsy model, as described herein.

Currently available therapeutic agents for blocking leukocyte recruitment include polypeptide therapeutics, e.g. antibodies, monoclonal antibodies, receptor-Fc chimeric fusion proteins, etc., and small molecule-based drugs. There are now multiple clinically validated anti-adhesion drugs. Small-molecule antagonists of adhesion molecule function can be categorized into three distinctive modes of action: ligand-mimetic competitive antagonists and allosteric antagonists or a I allosteric antagonists.

Approved therapies comprise an antibody fragment, ReoPro™, and two small-molecule inhibitors, Integrilin™ and Aggrastat™. These structures built on previously published structures of an integrin binding to its RGD based ligand. This information may yield additional routes to drug discovery that target medically relevant integrins.

The LFA-1/ICAM interaction is another key mediator of cell adhesion between leukocytes and vascular endothelium and, as both molecules are expressed on leukocytes, they are involved in modulating immune responses. In particular, targeting the integrin a chain (CD11a) has led to clinical success. The monoclonal antibody efaluzimab (Raptiva™) specifically recognizes the alpha chain of alphaLbeta2. It inhibits the ability of T cells to interact with Langerhans cells, endothelial cells and keratinocytes. Antisense specifically directed at ICAM-1 (Alicaforsen™) has also been developed and is in clinical trials. Small-molecule approaches have been under active research and has provided a variety of distinct antagonists in pre-clinical studies.

The leukocyte integrin .alpha.4.beta.1 interacts with its ligands VCAM and fibronectin. This is a key integrin-ligand interaction that allows leukocytes to adhere strongly to vascular endothelium and trigger subsequent shape changes in the leukocyte, ultimately leading to transmigration. Antibodies and small-molecule antagonists are effective in a wide range of animal models of inflammation. For example SB683699 for multiple sclerosis is being tested for inflammation and has demonstrated positive effects. The anti-VLA4 monoclonal antibody Tysabri/Antegren™ is also in use. Another VLA4 antagonist is CDP323, which is currently in clinical trials. Also in clinical trials is the immunoadhesin molecule YSPS.

“Target acquisition”, as used herein, refers to the successful interaction of a drug or biologic agent with the target cell or molecule. Target acquisition may be monitored by methods suitable for the specific interaction, e.g. a flow cytometry assay to look at saturation levels of an antibody on a target cell, calcium flux in a target population for a signaling molecule; and the like.

### Methods of the Invention

The methods of the invention are based, in part, on the finding of a role for neutrophils in the pathogenesis of AD, or other neuroinflammatory neurodegenerative diseases; and a demonstration that blockade of neutrophil adhesion; function; or interaction with the brain is useful in the treatment of neurodegenerative disease, including prevention of cognitive decline.

It is shown herein that there is an increase in the number of neutrophils in brains of AD patients compared to control age-matched subjects. Neutrophils in AD brains were adhered and spread on endothelial cells in cortical brain vessels and in the parenchyma, and are largely found in proximity to amyloid plaques, whereas the few neutrophils found in age-matched normal brains were confined to blood vessels.

Embodiments of the invention include methods to treat or prevent AD by any single or combination of the following methods: (i) depletion of neutrophil/myeloid cell populations systemically or locally in the brain; (ii) blocking neutrophils/myeloid cell adhesion and crawling; (iii) blocking transmigration and infiltration of neutrophils/myeloid cells into the brain; (iv) blocking cell-cell interactions between neutrophil/myeloid cells and endothelial cells and/or neural cells; (v) blocking neutrophil/myeloid cell extracellular-matrix interactions; (vi) reducing motility of neutrophils/myeloid cells in the brain parenchyma; (vii) blocking Aβ-induced activation and adhesion of neutrophils/myeloid cells; (viii) blocking intracellular signaling controlling adhesion and activation; (ix) blocking neutrophil activation and/or degranulation; (x) blocking release of reactive oxygen species, proteases, cytokines, lipid mediators or other damaging agents from myeloid cells and/or neutrophils; (xi) blocking neutrophil/myeloid cell activation leading to increased affinity and valency; (xii) blocking formation of neutrophil extracellular traps (NETS) in brain vessels or parenchyma.

In some embodiments the methods to block neutrophil/myeloid cells include but are not limited to: depletion of neutrophil/myeloid cell populations; blockade of formation of NETs; blockade of adhesion molecules, including integrins and their ligands, ICAM-1, LFA-1, CD11a, CD11b, CD11c, CD18; CD49; E-, P- and L-selectin and their ligands, including but not limited to PSGL-1, CD44, CD43, glycolipids; alpha-4 integrins and their ligands VCAM-1 and MAdCAM-1; CD44 and hyaluronan; protein tyrosine kinases, including but not limited to, Syk, Abl, JAK3, Jak 2, and BTK and MAPK; PI3K, and/or formyl peptide receptors.

One embodiment of the invention includes treatment of patients with AD, high risk of AD and/or other neuroinflammatory disease with an effective dose or dosing regimen of a therapeutic agent capable of blocking neutrophil and/or myeloid cell interaction with the brain vascular endothelium. While neutrophil invasion and migration within the AD brain parenchyma has been observed; the leukocyte interaction, including binding and spreading, with the vascular endothelium may be sufficient to cause significant pathology.

In some embodiments therapeutic agents target adhesion molecules, including but not limited to LFA-1 (CD11a and/or CD11b) and its ligand ICAM-1, to block neutrophil/myeloid cell binding, activation and/or invasion of the brain.

In some embodiments, therapeutic agents target alpha-4 integrin, VCAM-1, MAdCAM-1, the alpha-4-VCAM pathway and/or the alpha-4 integrin-MAdCAM-1 pathway to block neutrophil/myeloid cell binding, activation and/or invasion of the brain. Alpha-4 integrin is expressed on neutrophils under certain conditions and has been shown to be involved in certain human pathologies. Treatment with a neutralizing anti-alpha-4 antibody is shown herein to inhibit the development of cognitive deficit in models of AD.

Therapeutic agents targeting blockade of alpha-4 integrin-mediated T-cell recruitment have been developed to target treatment of multiple sclerosis (MS) and Crohn's disease. MS is an autoimmune disease with a prevalence of less than 0.015%. It afflicts predominantly women and the average of onset is approximately 34 years. Disease management includes immunosuppressive therapy including high doses of oral and/or intravenous corticosteroids, such as methylprednisolone. Crohn's disease is a type of inflammatory bowel disease with an incidence of 0.006%. The disease tends to strike people in their teens and twenties, and occasionally people over 50. Disease management often includes immunosuppressive therapy, including corticosteroids.

The MS and Crohn's disease patient populations are small, relatively young and highly exposed to immunosuppressive drugs. This patient population does not generally overlap with the AD patient population. Further, the broad, long-term exposure to immunosuppressants is a key distinguishing factor between these patient populations, since treatment with the anti-alpha-4 therapeutic antibody natalizumab in patients currently or previously treated with immunosuppressant drugs leads to increased risk of developing progressive multifocal leukoencephalopathy (PML), an often fatal opportunistic infection caused by the JC virus. No deaths have been linked to natalizumab when it was not combined with other immune-modulating drugs.

In some embodiments of the invention, individuals for treatment are differentially selected to exclude MS and Crohn's disease patient populations, based on current best practices of diagnosis as well as selected biomarkers defining AD patient populations. Target acquisition of the alpha-4 pathway in neutrophils in AD results in significantly different molecular, physiologic, and clinical changes compared to target acquisition of the alpha-4 pathway in T cells in MS or Crohn's disease.

In some embodiments of the invention, successful blockade of neutrophil/myeloid cell activation, interaction with the vascular endothelium in brain and/or invasion of brain in patients with AD is tracked using, for example, the numbers and/or activation status of circulating neutrophils, or for example one or likely multiple members of a signature molecular network shown to be dysregulated in AD.

One embodiment of the invention includes treatment of patients with AD, high risk of AD and/or other neuroinflammatory disease with an effective dose or dosing regimen of inhibitors, agonists or antagonists targeting ABL in patients selected for markers of AD not diagnosed with CML, ALL or other Philadelphia chromosome+ types of leukemia. The benefit of the invention is that the agent is not required to block BCR-ABL fusion proteins, imatinib-resistant ABL mutants or other ABL mutations. The class of ABL inhibitors targeting wild-type ABL in normal leukocytes in AD patients can be differentiated from the class of ABL inhibitors required to treat the mutated form of ABL in CML and other oncology indications.

Multiple inhibitors of ABL, including imatinib (market name Gleevec), dasatinib, nilotinib, bosutinib and GNF-2, can block Aβ-induced adhesion of human neutrophils, indicating that blockade of ABL represents a therapeutic strategy to prevent neutrophil/myeloid cell activation, interaction with vessels in the brain, and invasion of neutrophils into the brain. ABL is a well-described protein tyrosine kinase implicated in the processes of cell differentiation, cell division, cell adhesion, and stress responses. Mutations in ABL cause chronic myelogenous leukemia (CML). In CML the ABL gene is activated by chromosomal translocation or “switching” of parts of the 9^{th} and 22^{nd} chromosomes resulting in the fusing of ABL and BCR on chromosome 22. This new fusion gene, BCR-ABL, does not require normal activation signals and drives the cells to proliferate without regulation, i.e. the cells become cancerous. Multiple inhibitors for ABL are on the market and in the clinic targeting CML and other oncology indications. Second generation and third generation ABL inhibitors target imatinib-resistant mutants. Dasatinib can bind most imatinib-resistant mutants; however, this property reduces the specificity of the inhibitor resulting in cross-reactivity with other targets including the SRC family members. Thus, the class of ABL inhibitors required to treat CML can be differentiated from inhibitors targeting the wild-type gene found in normal leukocytes. Further, the dose and regimen can also be differentiated.

The patient population targeted for ABL inhibitor therapy for AD would be non-overlapping, and differentially selected and tracked compared to the patient population targeted for treatment for CML. In the CML population, drug effectiveness and cytogenic remission are tracked by screening for absence of t (9,22) translocation in bone marrow metaphase cells or by PCR screening for BCR-ABL transcripts.

Target acquisition of ABL in AD results in significantly different molecular, physiologic, and clinical changes compared to target acquisition in CML. In some embodiments of the invention, target acquisition of ABL in normal neutrophils/myeloid cells in patients with AD or other neuroinflammatory disease is tracked by a range of possible biomarkers and clinical symptoms, for example, the numbers and/or activation status of circulating neutrophils, inflammatory markers in the blood and/or cerebrovascular fluid, for example one or likely multiple members of a signature molecular network shown to be dysregulated in AD. The molecular network includes specific cytokines, chemokines and growth factors that have been reported to have different expression in patients with neurological disease (Britschgi M and Wyss-Coray, T, 2007, Int. Rev. Neurobiol 8, 205-233; Britschgi M et al., 2011, Mol Cell Proteomics. October; 10(10):M111). The standard measures to evaluate AD patients could also be used, including cognitive and behavioral screening, positronic emission tomography (PET) amyloid imaging, CSF levels of Aβ, and phosphorylated tau, and brain imaging (structural).

Ibrutinib, a selective inhibitor of BTK, is a potent inhibitor of Aβ-induced adhesion of human neutrophils, providing a therapeutic strategy to prevent neutrophil interaction with vessels in the brain and invasion of neutrophils into the brain. One embodiment of the invention includes treatment of AD patients with a dose or dosing regimen of pharmaceutical agents that block Bruton's tyrosine kinase (BTK) activity to block neutrophil/myeloid cell activation, binding and/or invasion of the brain. In this embodiment, patients are selected for the molecular profile of AD, which is substantially differentiated from selection of patients with B-cell malignancies including but not limited to chronic lymphocytic leukemia, mantle cell lymphoma, diffuse large B-cell lymphoma and multiple myeloma.

BTK is a non-receptor tyrosine kinase and plays a central role in B cell receptor (BCR) signaling. Upon BCR activation, BTK becomes activated by other tyrosine kinases resulting in B cell proliferation and differentiation. BTK is also involved in B cell migration and adhesion. Because of its prominent role in B cell development and function, BTK became the target for therapeutics in autoimmune disease, such as rheumatoid arthritis and B cell malignancies. The BTK inhibitor ibrutinib has emerged as a breakthrough in targeted therapy for certain types of B cell malignancies, including chronic lymphocytic leukemia, mantle cell lymphoma, diffuse large B-cell lymphoma and multiple myeloma. It also has potential effects against autoimmune arthritis.

The patient population with B-cell malignancy and autoimmune disease are substantially different from the AD population. CLL is the most common type of adult leukemia, with an incidence of less than 0.005% afflicting mostly men with a median age of diagnosis of 72 yrs. Mantle cell lymphoma, a rare form of non-Hodgkin's lymphoma, is quite rare (there are approximately 15,000 patients in the US in 2012), also occurring mostly in men. Multiple myeloma occurs in less than 0.005% of the population is also more common in men, with a median age of diagnosis of 69 years. Autoimmune arthritis strikes approximately 0.6% of the US population, occurring more frequently in women. Although the age range for B-cell malignancy and autoimmune disease overlap somewhat with the AD population, the gender skew and small population mean that overall, the patient populations are largely differentiated. In 2013 over 5 million Americans are living with AD, including an estimated 200,000 under the age of 65.

Since interruption of BCR signaling in B cells is the target for BTK inhibitors in B-cell diseases, target acquisition by ibrutinib or other BTK inhibitors in B-cell malignancy is demonstrated in patients by redistribution of tissue-resident CCL cells into the blood with rapid resolution of enlarged lymph nodes and eventually normalization of lymphocyte counts. Clinical endpoints include progression-free survival and duration of response.

Target acquisition of BTK in neutrophils/myeloid cells in AD results in significantly different molecular, physiologic, and clinical changes compared to target acquisition in B-cell malignancy and autoimmune disease.

One embodiment of the invention provides treatment of patients with AD, high risk of AD and/or other neuroinflammatory disease with an effective dose or dosing regimen of a pharmaceutical agent that blocks spleen tyrosine kinase (SYK) to block neutrophil/myeloid cell activation, binding and/or invasion of the brain. SYK inhibitors piceatannol and PRT are potent inhibitors of Aβ-induced adhesion of human neutrophils, indicating that blockade of SYK is a therapeutic strategy to prevent neutrophil interaction with vessels in the brain and invasion of neutrophils into the brain.

SYK is a non-receptor tyrosine kinase expressed in hematopoietic tissue and plays a key role in signaling from the BCR in B cells and the T-cell receptor (TCR) in T cells. SYK can also transmit signals from other cell surface receptors including the Fc receptor and integrins. Abnormal function of Syk has been implicated in several hematopoietic malignancies including translocations involving IL-2-inducible T cell kinase (ITK), expressed in T cells, and ETV6, an oncogene expressing the ETCS family transcription factor.

SYK is a drug target for autoimmune disease, such as RA and SLE, as well as hematological cancers including chronic lymphoid leukemia, multiple myeloma and non-Hodgkin lymphoma as well as asthma. The patient populations for these indications are differentiated from the AD patient population, and target acquisition in AD in neutrophils and MYELOID cells is also distinct, as described above for BTK.

One embodiment of the invention provides treatment of patients with AD, high risk of AD and/or other neuroinflammatory disease with an effective dose or dosing regimen of a pharmaceutical agents that blocks JAK3 to block neutrophil/myeloid cell activation, binding and/or invasion of the brain. Multiple inhibitors of JAK3, AG490, WHI-P145, P1-TKIP, CP-690550, ruxolitinib and TG1011348, can block Aβ-induced adhesion of human neutrophils, indicating that blockade of JAK3 represents a therapeutic strategy to prevent neutrophil interaction with vessels in the brain and invasion of neutrophils into the brain.

JAK has been identified as a target in a broad range of indications including RA, myelofibrosis, autoimmune disorders, solid tumors, hematological cancers, myeloproliferative disorders, pancreatic cancer, kidney transplant rejection, ulcerative colitis, dry eye disease and Crohn's disease. The dosing and regimen for targeting JAK in neutrophils can be differentiated from targeting JAK in T and B cells, and target acquisition in neutrophils and MYELOID cells in AD is also distinct, as described above for BTK.

Aβ₁₄₂ oligomers induce rapid adhesion of mouse and human neutrophils to fibrinogen, and the FPR antagonist boc-MLF inhibits Aβ-induced neutrophil adhesion. In addition, pertussis toxin (PTx) also abrogates Aβ-induced neutrophil adhesion. Together these data show FPR as a binding site for Aβ on neutrophils, providing for FPR as a therapeutic target for treatment of AD and other neurodegenerative disease.

One embodiment of the invention includes treatment of patients with AD, high risk of AD and/or other neuroinflammatory disease with an effective dose or dosing regimen of pharmaceutical agents that block FPR to block neutrophil/myeloid cell activation, binding and/or invasion of the brain. This invention offers the advantage that in this embodiment blockade of FPR in neutrophils/myeloid cells does not require crossing the BBB. A number of peptide and non-peptide agonist and antagonist inhibitors have been identified targeting FPR on neuonal cells and targeting the inflammatory glia cytokine pathway in glial cells; however, drug delivery to the central nervous system is notoriously difficult by restrictive mechanisms imposed at the blood-brain barrier. Targeting FPR on circulating neutrophils and other myeloid cells is used in treating Alzheimer's disease and is differentiated from targeting FPR on resident glial cells in the brain, both by target cell type, drug delivery location, regimen and dosing requirements for target acquisition.

The blood brain barrier (BBB) is formed by specialized tight junctions between the epithelial cells that surround brain tissue and serves to prevent many large molecules and some ions to pass into the brain. The BBB limits entry of many therapeutic agents even when the BBB is compromised to some extent. There are some promising new approaches focused on solving the BBB-drug delivery challenge; however, therapeutics and treatment approaches that obviate the need to cross the BBB offer distinct advantages. FPR agonist and antagonist inhibitors that are not able to cross the BBB would be sufficient to target circulating neutrophils/myeloid cells and would not be candidates for targeting microglia in the brain.

Some embodiments of the invention include administration of agents that target serine/threonine kinases, including but not limited to, PKC; lipid mediators and lipoxins; chemokine receptors; proinflammatory cytokines such as TNFα; proinflammatory transcription factors in neutrophil/myeloid cells including but are not limited to NF-κB; inhibitors of oxygen burst and/or degranulation and the toxic compounds in neutrophil granules, including but not limited to matrix metalloproteinases and NADPH; neutrophil survival factors, including but not limited to GM-CSF, G-CSF; interleukins including but not limited to IL-1alpha, IL-1 beta and IL-6. In these embodiments, these strategies are focused on blocking neutrophil/myeloid cell activation, function, adhesion, diapedesis, ROS release and/or migration in brain to prevent cognitive decline in AD and other neurodegenerative disease.

In some embodiments of the invention, pharmacologic agents targeting neutrophil/myeloid cell activation, adhesion and/or invasion of the brain to prevent or treat cognitive decline are administered at early, mid and/or late stages of the disease and can be administered intermittently at early, mid and/or late stages of disease. Cognitive decline in AD is not correlated with the level of Aβ accumulation and deposition in the brain. It has been suggested that once a threshold of Aβ accumulation is reached sufficient to trigger inflammation and neuronal toxicity, additional Aβ accumulation and possibly removal is not a factor in disease progression. That along with the failure of drug trials targeting Aβ in mild-moderate AD patient populations has led to the suggestion Aβ targeted interventions will yield clinical benefit only if they are initiated very early, before the threshold is met. In contrast, neuroinflammation is an ongoing process and therefore amenable to treatment over the course of the disease.

Here we show that early-mid stage, short-term blockade of neutrophil presence, activation, function and adhesion results in immediate as well as long-term protection against cognitive decline. Ongoing or intermittent neutrophil depletion, blockade of neutrophil activation, adhesion and/or migration into the brain can be beneficial at early and later stages of disease.

In some embodiments of the invention, administration of pharmacological agents targeting blockade of neutrophil/myeloid cell activation, function, adhesion, diapedesis, ROS release and migration in brain results in normalization of Aβ levels and/or normalization of tau phosphorylation and/or reversal/prevention of cognitive decline in AD and other neurodegenerative disease. Here we show that blockade of neutrophil/myeloid cells results in reversal/prevention of cognitive decline, normalization of tau phosphorylation and normalization of Aβ levels. Therapeutic strategies capable of simultaneously affecting accumulation of AD and/or multiple AD-induced neurodegenerative mechanisms seem more likely to be efficacious. In addition, the combination of removal or reduction of the inflammatory trigger and blunting of the inflammatory response to the trigger, in a single therapeutic or with a combination of therapeutics, may provide a superior approach.

In the methods of the invention, leukocyte concentration or activity in the brain is modulated through administering compounds that are agonists or antagonists of specific targeting pathways, including without limitation those pathways involved in leukocyte trafficking and/or activation. Antagonists include, for example, agents that interfere with the interaction between integrins involved in neutrophil trafficking and their ligands; agents that interfere with neutrophil activation; and agents that interfere with neutrophil chemotaxis, which include, without limitation, antibodies specific for specific targets in these pathways. The methods of the invention are used to promote an improved outcome in patients suffering from neurodegenerative disorders, e.g. Alzheimer's disease, etc.

The agonists and/or antagonists of the present invention are administered at a dosage that modulates neutrophil concentration or activity while minimizing any side-effects. It is contemplated that compositions will be obtained and used under the guidance of a physician for in vivo use. The dosage of the therapeutic formulation will vary widely, depending upon the nature of the disease, the frequency of administration, the manner of administration, the clearance of the agent from the host, and the like.

The effective amount of a therapeutic composition to be given to a particular patient will depend on a variety of factors, several of which will be different from patient to patient. Utilizing ordinary skill, the competent clinician will be able to optimize the dosage of a particular therapeutic or imaging composition in the course of routine clinical trials.

Therapeutic agents, e.g. agonists or antagonists can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intrathecal, nasal, intracheal, etc., administration. The active agent may be systemic after administration or may be localized by the use of regional administration, intramural administration, or use of an implant that acts to retain the active dose at the site of implantation.

One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents is also an option. A BBB disrupting agent can be co-administered with the therapeutic compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. Alternatively, drug delivery behind the BBB is by intrathecal delivery of therapeutics or imaging agents directly to the cranium, as through an Ommaya reservoir.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure, e.g. intravascular (intravenous, intraarterial, intracapillary) administration, injection into the cerebrospinal fluid, intracavity or direct injection in the brain. Intrathecal administration may be carried out through the use of an Ommaya reservoir, in accordance with known techniques. (F. Balis et al., Am J. Pediatr. Hematol. Oncol. 11, 74, 76 (1989).

Where the therapeutic agents are locally administered in the brain, one method for administration of the therapeutic compositions of the invention is by deposition into or near the site by any suitable technique, such as by direct injection (aided by stereotaxic positioning of an injection syringe, if necessary) or by placing the tip of an Ommaya reservoir into a cavity, or cyst, for administration. Alternatively, a convection-enhanced delivery catheter may be implanted directly into the site, into a natural or surgically created cyst, or into the normal brain mass. Such convection-enhanced pharmaceutical composition delivery devices greatly improve the diffusion of the composition throughout the brain mass. The implanted catheters of these delivery devices utilize high-flow microinfusion (with flow rates in the range of about 0.5 to 15.0 μl/minute), rather than diffusive flow, to deliver the therapeutic composition to the brain and/or tumor mass. Such devices are described in U.S. Pat. No. 5,720,720, incorporated fully herein by reference.

The effective amount of a therapeutic composition to be given to a particular patient will depend on a variety of factors, several of which will be different from patient to patient. A competent clinician will be able to determine an effective amount of a therapeutic agent to administer to a patient. Dosage of the agent will depend on the treatment, route of administration, the nature of the therapeutics, sensitivity of the patient to the therapeutics, etc. Utilizing LD₅₀ animal data, and other information, a clinician can determine the maximum safe dose for an individual, depending on the route of administration. Utilizing ordinary skill, the competent clinician will be able to optimize the dosage of a particular therapeutic composition in the course of routine clinical trials. The compositions can be administered to the subject in a series of more than one administration. For therapeutic compositions, regular periodic administration will sometimes be required, or may be desirable. Therapeutic regimens will vary with the agent, e.g. some agents may be taken for extended periods of time on a daily or semi-daily basis, while more selective agents may be administered for more defined time courses, e.g. one, two three or more days, one or more weeks, one or more months, etc., taken daily, semi-daily, semi-weekly, weekly, etc.

Formulations may be optimized for retention and stabilization in the brain. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject invention. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

The invention may be better understood with reference to the accompanying examples.

### Experimental

The endothelial integrin ligand VCAM-1 and the selectins E- and P-selectin are expressed by endothelial cells in blood vessels, including meningeal vessels and first layers of the cortex and hippocampal vessels of 5XFAD and 3xTG mouse models during early phases of disease of Alzheimer's disease and are not expressed in normal brain. Neutrophils/myeloid cells were observed (using immunohistochemistry of sections) adhered inside blood vessels and migrated into the parenchyma mainly in the meninges in deep cortical layers, and also in the choroid plexus and in close proximity to the hippocampus and amygdala in both mouse models of AD but not in normal healthy mice. GR1+ cells, quantitated using flow cytometry, were increased in AD mice as early as 2 months, peaked at approximately 4 months in 5XFAD mice and 5-6 months in the 3xTG model, and declined gradually but remained well above the levels observed in normal mice out to the last time-point observed (10 months).

We describe for the first time the behavior of neutrophils inside cerebral vessels and inside CNS parenchyma in animal models of AD. Intravital TMP was used to observe the behavior (interaction with the vascular endothelium and motility in the tissue) of bone-marrow-derived neutrophils labeled with a fluorescent dye injected into normal and 5xFAD mice. Neutrophils rolled along the endothelium, firmly adhered, crawled and extravasated into the brain parenchyma exclusively in areas with amyloid plaques and displayed a highly motile phenotype in the 5XFAD mice. Moreover, neutrophils displayed immediate arrest, adhesion and intraluminal crawling inside vessels with amyloid deposition, indicating that Aβ is involved in neutrophil entry into the brain parenchyma. In contrast, there was no evidence of adhesion or extravasation in age-matched wild-type control mice.

We also found that the soluble oligomeric Aβ are able to induce NADPH oxidase activation in mouse and human neutrophils, indicating that ROS production induced by soluble Aβ oligomers may represent a mechanism of neutrophil-dependent injury in AD and therefore a target for blockade of neurotoxicity.

Central to the invention is the discovery that short-term (4-6 weeks) depletion of GR1+ cells at the mid-stage disease, after behavioral changes are observed (month 4 in the 5XFAD model, ˜month 6 in the 3xTG model) resulted in a profound blockade of cognitive decline that was maintained many months later in aged mice. At the end of the treatment period, control-antibody treated AD mice showed significant cognitive impairment in two standard tests (the Y-maze spontaneous alternation task to measure special working memory and the contextual fear conditioning to measure hippocampus-dependent form of memory). In contrast, GR1+ cell-depleted mice showed no cognitive impairment, performing equally well in both tests compared to age-matched normal healthy mice. It is important to note that treatment was initiated after behavioral changes were evident. The 3xTG mice were tested also 6 months after the treatment termination and at 13 months of age. The control-antibody-treated mice showed significant further cognitive decline, as expected in this model, whereas the GR-1+ depleted mice showed no cognitive impairment compared to healthy, normal age-matched mice in the fear conditioning test and significantly better cognitive function in the Y-maze test, compared to the control-antibody-treated mice. Thus, short-term blockade of GR-1+ cell interaction with, and binding to, brain vascular endothelium and entry into the brain resulted restored cognitive function, prevention of cognitive decline and long-term preservation of cognitive function.

LFA-1 is a key integrin involved in neutrophil arrest and ligation of specific GPCRs by chemokines and other chemoattractants triggers an increase in LFA-1 affinity and valency. We discovered that Aβ₁₋₄₂ oligomers trigger the LFA-1 conformation to an intermediate- and high-affinity state in human neutrophils, indicating that Aβ enhances the ability of LFA-1 to bind its endothelial ligand, and likely drives the surprising capacity of neutrophils to arrest in areas with amyloid deposition in vivo.

Another key element of the invention is the demonstration that therapeutic treatment AD mice with anti-adhesion MAbs anti-LFA-1 and anti-alpha4 integrin at the mid-stage of disease resulted in blockade of cognitive decline and preservation of cognitive function. Short-term (6 week) therapeutic treatment with anti-LFA-1 MAb starting at 6 months, at the mid-stage of disease in 3xTG AD mice (5.5-6 months, after behavioral changes are observed), preserved cognitive function measured in two tests at 13 months. In addition, short-term (6 week) therapeutic treatment with anti-alpha4 Mab starting at 6 months in 3xTg mice resulted in preservation of cognitive function to almost that of wild-type, age-matched control mice.

In addition, depletion of GR1+ cells also blocked accumulation of Aβ and phosphorylation of tau in the brains of 3xTG mice. Control antibody-treated 3xTG mice had elevated levels of Aβ and phosphorylated tau compared to wild-type control mice (as previously shown for this model). Surprisingly, depletion of GR1+ cells reversed amyloid deposition and reduced tau phosphorylation to the level of healthy control mice. This discovery demonstrates a role for GR1+ cells in amyloid deposition and induction of tau phosphorylation, two pivotal mechanisms in the induction of behavioral impairment in AD-like disease, and suggests that inhibition of neutrophil function may represent a new therapeutic approach in AD.

Since Aβ is able to induce neutrophil adhesion in vitro and neutrophils/myeloid cells found in both mouse and human AD brains are largely localized to areas of Aβ deposition, Aβ-induced neutrophil adhesion and extravasation appears to be a key target for treatment of AD. We demonstrate that inhibitors to protein tyrosine kinases involved in GPCR signaling leading to activation and adhesion (JAK, ABL1, BTK and SYK) are able to prevent neutrophil adhesion triggered by Aβ, thus, blockade of neutrophil function and adhesion using inhibitors of signaling represent an additional approach for treatment of AD.

### Example 1

### Vascular Adhesion Molecules ICAM-1, VCAM-1, E and P-Selectin are Expressed at Early Stage of Disease in 5XFAD and 3xTg Mice

Adhesion molecules are fundamental mediators of leukocyte recruitment in sites of inflammation. We demonstrated expression of vascular adhesion molecules in the brain of 5XFAD and 3xTG mouse models of AD. We used APP/PS1 double transgenic mice with five FAD mutations (5XFAD) that co-express high transgene levels and co-inherit both APP and PS1 (Oddo S, et al, 2003, Neuron 39:409-421). The genetic backgrounds of 5XFAD mice were 50% C57Bl/6 and 50% SJL. Transgenic lines were maintained by crossing heterozygous transgenic mice with B6SJL F1 breeders. All transgenic mice used were heterozygotes with respect to the transgene, and non-transgenic littermates served as controls. Genotyping was performed by polymerase chain reaction analysis of tail DNA. 3xTg-AD mice were previously obtained by co-microinjecting two independent transgenes encoding human APPSwe and the human tauP301 L (both under control of the mouse Thy1.2-regulatory element) into single-cell embryos harvested from homozygous mutant PS1M146V knock-in (PS1-KI) mice. 5XFAD and 3xTg-AD mice were purchased from ‘The Jackson Laboratory’ (Sacramento, Calif.). Animals were housed in pathogen-free climate controlled facilities and allowed to have food and water ad libitum.

We performed confocal microscopy studies to evaluate the expression of integrin ligands, such as VCAM-1 and ICAM-1 and selectins such as E- and P-selectin on brain sections of 5XFAD mice. Frozen tissues stored with OCT compound were cryo-sectioned. Mouse brains were cut in coronal slices of 30-40 μm. Sections were collected and placed in 24-well plates containing 1 ml of PBS. Then, free floating sections were incubated in blocking buffer, corresponding to species for secondary Aβ, for 1 hour at room temperature; then treated with primary antibody overnight at 4° C. (anti-VCAM at the concentration of 40 μg/ml, anti-ICAM 10 μg/ml, anti-P-selectin 10 μg/ml, anti-E-selectin 5 μg/ml, anti-CD45 5 μg/ml, anti-CD11c at 1 μg/ml, anti-CD3 5 μg/ml, anti-GR1 5 μg/ml and anti-F4/80 1 μg/ml). Sections were rinsed with PBS and at last stained for 3 minutes at RT° C. with 0.1% of filtered Thioflavin S solution. Slices were incubated with biotinylated secondary antibody (rabbit anti-rat, T0226, VectorLab and goat anti-hamster, BA-9100, VectorLab, both at the final concentration of 7.5 μg/ml) for 1 h at RT° C., then washed with PBS. To reveal immunostaining, slices were treated with Avidin Texas Red (at the final concentration of 25 μg/ml, A2006, Vector Lab) for 1 h at RT° C. in the dark. After rinsing in PBS, sections were incubated with Dapi (at the final concentration of 1 μg/ml, D9542, Sigma-Aldrich, St. Louis, Mo.), for 8 min in the dark. Finally, brain portions were washed with PBS, transferred on glass slides and mounted with medium Dako (S3023, DAKO NORTH AMERICA, Carpinteria, Calif.). Slides were analyzed by Tandem Confocal Scanning-SP5 (Leica, Germany).

At 2 months of age the adhesion molecule expression was slightly increased compared to age-matched controls, preferentially in meningeal vessels and first layers of the cortex. At 4 months of age all studied adhesion molecules were found highly expressed in meningeal vessels, first layers of the cortex, but also in hippocampus and amygdala compared to age-matched controls (Table 1). Interestingly, the increase in adhesion molecules expression was found in cerebral vessels of the cortex in close proximity to amyloid angiopathy and/or parenchymal Aβ plaque depositions. To confirm the results obtained with the 5XFAD model, we next studied the expression of endothelial adhesion molecules in 3xTg mice. Confirming the data obtained in 5XFAD mice, we found an increased expression of all adhesion molecules, particularly of E- and P-selectin, mainly in the hippocampus at early stages of disease at 5 months of age, and 6 months of age, compared to age-matched controls (Table 1). These results show that vascular endothelium is inflamed in mice with cognitive deficit during early stage of disease and may potentially mediate leukocyte trafficking in the CNS.

TABLE 1
Expression of vascular adhesion molecules in brain of 5XFAD and
3xTgAD mice at early stage of disease

| | ICAM-1 | VCAM-1 | E-selectin | P-selectin |
|---|---|---|---|---|
| WT ctrl (B6SJL) | +/−^{a} | −^{a} | −^{a} | −^{a} |
| 5xFAD | +^{a} | +^{a} | +^{a} | +^{a} |
| WT ctrl (C57/BI6) | +/−^{b} | −^{b} | −^{b} | −^{b} |
| 3xTgAD | +^{b} | +^{b} | +^{b} | +^{b} |

^{a}meningeal vessels (mainly in the vessels of the meninges and cortex, but also in the choroid plexi, hippocampus and amygdala)
^{b}hippocampal vessels (especially E-selectin and P-selectin in the hippocampus and cortex)
Expression of vascular adhesion molecules in brain of 5XFAD and 3xTgAD mice at the early stage of disease. Confocal microscopy showed expression of endothelial integrin ligands ICAM-1 and VCAM-1; and selectins (E-selectin and P-selectin) in 30 μm coronal brain sections of healthy control B6SJL and C57BL/6 mice and 5XFAD and 3xTgAD mice. Increased expression of adhesion molecules was found mainly in meningeal vessels of 5XFAD mice (e, ICAM; f, VCAM; g, E-selectin; h, P-selectin) versus control at 4 months of age compared to age-matched controls. Basal expression of ICAM-1 was found in meningeal vessels of healthy mice. High expression of E- and P-selectin and moderate expression of ICAM-1 and VCAM-1 was found in hippocampal vessels at 6 months old 3xTgAD mice compared to healthy controls.

### Example 2

### Aβ1-42 Oligomers Induce Expression of Adhesion Molecules on Bend.3 Brain Endothelial Cell Lines

Emerging data suggest that soluble oligomeric Aβ forms rather than fibrillar Aβ are the amyloid species associated with AD neuropathology and cognitive dysfunction. We performed in vitro experiments to study the effect of soluble Aβ₁₋₄₂ oligomers on the endothelial cell line Bend.3, derived from mouse cerebral cortex purchased by ATCC& Cell Biology Collection (CRL-2299TM).

Briefly, Bend.3 were cultured at a concentration of 20×10³/ml on a glass coverslip in 24-well plates containing DMEM supplemented with 10% Fetal Calf Serum (FCS). Cells were stimulated with 10 μM Aβ 1-42, for 18 h in DMEM with 1% FCS. 47 Aβ positive control, cells were treated with 25 U/ml of TNFα for 18 h in DMEM with 1% FCS. Bend.3 were rinsed with PBS and fixed with 4% PFA for 10 minutes. Cells were washed with PBS incubated with blocking buffer for 1 h at RT° C. Primary antibodies were diluted in 1% Bovine Serum Albumin (BSA) PBS. Cells were incubated overnight at 4° C. with 50 μg/ml anti-VCAM (hybridoma MK2.7), 10 μg/ml anti-ICAM (hybridoma YN1.7), 0.5 μg/ml anti-CD31 (370700, Invitrogen), 5 μg/ml anti-E-selectin (hybridoma RME-1) and 1.5 μg/ml anti-P-selectin (CD62P, BD Pharmigen). The day after, cells were rinsed 2 times with PBS and then incubated with 7.5 μg/ml biotinylated secondary antibody (rabbit anti-rat, T0226, VectorLab) for 1 h at RT° C. Cells were rinsed 2 times with PBS and incubated with 25 μg/ml Avidin-TexasRed (A2006, Vector Lab) for 1 h at RT° C. in the dark. After rinsing in PBS, cells were incubated with 1 μg/ml Dapi (D9542, Sigma-Aldrich, St. Louis, Mo.), for 5 minutes in the dark. Finally, glass coverslip was transferred on glass slides and mounted with Dako medium (S3023, DAKO NORTH AMERICA, Carpinteria, Calif.). Glass slides were kept at 4° C. in the dark and acquired by LEICA fluorescence microscopy (DM6000B, LEICA, Germany).

We analyzed the expression of VCAM-1, ICAM-1, E- and P-selectin on Bend.3 after Aβ1-42 oligomers stimulation. TNF-α stimulation, which is well known to up-regulate the adhesion molecules expression, was used as positive control. We evaluated a dose-response effect of Aβ1-42 soluble oligomers on Bend.3 cells, ranging from 1 to 10 μM after 6 or 18 hours of treatment. After 18 h of treatment with 10 μM Aβ we observed a significant up-regulation of VCAM-1, ICAM-1, E- and P-selectin expression compared to non-stimulated control cells (CTRL) (Table 2). VCAM-1 and selectins were not expressed (indicated by -, Table 2) at basal condition on Bend.3 cells, and Aβ1-42 treatment at 10 μM highly up-regulated these molecules (Table 2). ICAM-1 was constitutively expressed at low levels on endothelial cells (indicated by +/−, some vessels were positive for ICAM-1, Table 2), but 18 h treatment with Aβ1-42 led to an increase of ICAM-1 expression (indicated by +, Table 2). These results show that soluble oligomeric Aβ directly activates endothelial cells and induces upregulation of adhesion molecules potentially allowing leukocyte adhesion.

TABLE 2
Soluble Aβ₁₋₄₂ oligomers up-regulates adhesion molecules on Bend.3
endothelial cell line. In vitro experiments on mouse brain
endothelial cell line Bend.3

| | Ctrl | Abeta 1-42 | TNF-a |
|---|---|---|---|
| ICAM-1 | +/− | + | + |
| VCAM-1 | − | + | + |
| E-sel | − | + | + |
| P-sel | − | + | + |
| CD31 | + | + | + |

Soluble Aβ₁₋₄₂ oligomers up-regulates adhesion molecules on Bend.3 endothelial cell line. In vitro experiments on mouse brain endothelial cell line Bend.3 were performed to examine the effect of soluble Aβ 1-42 oligomers on the expression of adhesion molecules (ICAM-1, VCAM-1) and selectins (E- and P-selectin). The effect was evaluated after 18 h treatment at the concentration of 10 μM Aβ₁₋₄₂ oligomers. Immunofluorescence staining allowed demonstration of up-regulation of ICAM-1, VCAM-1, E-selectin and P-selectin expression, when compared to control Bend.3 cells, which were treated with the buffer used for Aβ₁₋₄₂ oligomers. ICAM-1 was constitutively expressed at low levels on endothelial cells. TNFα stimulation, which is known to up-regulate the expression of vascular adhesion molecules, was used as positive control. CD31 was used as typical endothelial cell marker. A secondary antibody staining was used to verify the lack of aspecific fluorescence signal on cells.

### Example 3

### GR1+ Leukocytes Migrate into the Brain During Early Disease

To check whether vascular adhesion molecules may mediate leukocyte trafficking during early disease, we performed confocal microscopy experiments to seek for the presence of leukocyte infiltration into the brain parenchyma. The inflammatory cells were detected on 5XFAD brain slices using antibodies towards specific cell surface antigens, including CD45 as a general leukocyte marker, CD3 for T lymphocytes, CD11c for monocytes, and F4/80 for macrophages. Gr1 staining for neutrophils was performed with RB6-8C5 antibody.

Frozen sections were prepared as described above. Anti-CD45 5 μg/ml, anti-CD11c at 1 μg/ml, anti-CD3 5 μg/ml, anti-GR1 5 μg/ml and anti-F4/80 1 μg/ml). Sections were rinsed with PBS and at last stained for 3 minutes at RT° C. with 0.1% of filtered Thioflavin S solution. Slices were incubated with biotinylated secondary antibody (rabbit anti-rat, T0226, VectorLab and goat anti-hamster, BA-9100, VectorLab, both at the final concentration of 7.5 μg/ml) for 1 h at RT° C., then washed with PBS. To reveal immunostaining, slices were treated with Avidin Texas Red (at the final concentration of 25 μg/ml, A2006, Vector Lab) for 1 h at RT° C. in the dark. After rinsing in PBS, sections were incubated with Dapi (at the final concentration of 1 μg/ml, D9542, Sigma-Aldrich, St. Louis, Mo.), for 8 min in the dark. Finally, brain portions were washed with PBS, transferred on glass slides and mounted with medium Dako (S3023, DAKO NORTH AMERICA, Carpinteria, Calif.). Slides were analyzed by Tandem Confocal Scanning-SP5 (Leica, Germany).

In agreement with the results showing expression of vascular adhesion molecules, we observed higher numbers of CD45+ cells in the brain during early stages of disease in 5XFAD mice compared to age-matched littermates, mainly localized in meningeal vessels and within or around cortical vessels at 4 months of age. Surprisingly, at this early stage of disease we observed numerous Gr1+ cells adhered inside blood vessels or migrated inside parenchyma mainly in the meninges in deep cortical layers (Table 3), but also in choroid plexi and in close proximity to hippocampus and amygdala. As observed for adhesion molecules expression, cell infiltration was found in vessels of the cortex in the vicinity of amyloid depositions. Confirming the data obtained in the 5XFAD mouse model, we observed CD45 and Gr1 double positive cells at early stages of disease at 5 months of age and 6 months of age also in six months old 3xTg mice (Table 3) when compared to healthy mice Cell infiltration in this AD mouse model was mainly localized in the choroid plexi and hippocampus.

TABLE 3
Neutrophils adhere in brain vessels and migrate into the parenchyma of
5xFAD and 3XTgAD mice early in disease.

| | CD45 | CD45 | Gr1 | Gr1 |
|---|---|---|---|---|
| WT ctrl (B6SJL) | −^{a} | −^{a} | −^{a} | −^{a} |
| 5xFAD (5 months) | +^{a} | +^{a} | +^{a} | +^{a} |
| WT ctrl (C57BL/6) | −^{b} | −^{b} | −^{b} | −^{b} |
| 3xTg-AD (6 months) | +^{b} | +^{b} | +^{b} | +^{b} |

^{a}meningeal vessels and cortical vessels
^{b}choroid plexi and hippocampus
Neutrophils adhere in brain vessels and migrate into the parenchyma of 5XFAD and 3XTgAD mice during early disease. Confocal microscopy images show the presence of CD45+ leukocytes, and Gr1+ cells localized in meningeal vessels and in deep cortical layers of B6SJL control animals versus 5XFAD mice at 4 months of age, and in the plexi of lateral ventricles and hippocampal parenchyma in C57BL/6 healthy controls versus 3xTgAD mice of 6 months of age. 8-10 animals/group of same age were analyzed for both AD
models.

We next quantified the accumulation of leukocytes from the brains of 5XFAD and 3xTg mice using flow cytometry. Briefly, mice were anesthetized and perfused through the left cardiac ventricle by injection of 35 ml of cold PBS. The brain was dissected, cut into small pieces and digested with DNasel (20 U/ml, Invitrogen) and collagenase (1 mg/ml, Sigma) at 37° C. for 45 minutes. Cells were isolated by passing the digested tissue through a cell strainer (70 μm), resuspended in 30% percoll and loaded onto 70% percoll. Tubes were then centrifuged at 1300×g for 20 minutes at 4° C. Cells were removed from the interphase, washed twice in PBS and resuspended in staining buffer for further analysis. Sensitive identification of various immune cell populations in a single sample was performed by antibody staining and flow cytometry with MACSQuant Analyzer (Miltenyi Biotec, Germany). The following anti-mouse antibodies were used: anti-CD45-Vioblue, anti-CD11 b-FITC, anti-Gr1-PE, anti-Ly6G-APC (Miltenyi Biotec, Germany) and anti-CD3E (Biolegend, San Diego, Calif., USA). Data were analyzed using FlowJo software.

To quantify neutrophil population, CD45 positive cells were sub-gated by using CD11 b and Gr1 double gate allowing the identification of 3 different populations: CD11 b/Gr1 negative cells, CD11 b positive/Gr1 negative cells (presumably microglia) and CD11 b positive/Gr1 positive cells (presumably granulocytes). For a more specific polymorphonuclear leukocyte (PMN) labeling, Gr1 and Ly6G (staining with 1A8 antibody) sub-gate was also performed during analysis.

The results showed that the number of infiltrating neutrophils peaked at approximately 4 months of age in 5XFAD mice and at 5-6 months of age in the 3xTg model and then descended gradually in subsequent months (Table 4; mean+/−SEM), but were always present in significant numbers. Almost no neutrophils were detected in brains of healthy controls. Taken together these data show that neutrophils start to migrate into the brain in the early stage of disease and continue to accumulate throughout the phases of disease, suggesting they may play a role during early disease as well as in chronic disease evolution.

TABLE 4
Timeline of neutrophil/myeloid cell infiltration in the brain of 5xFAD and 3xTG models of AD

| 5X-FAD | WT | | 5xFAD | |
|---|---|---|---|---|
| Months of Age | CD11b+/Gr1+ | Gr1+/Ly6G+ | CD11b+/Gr1+ | Gr1+/Ly6G+ |
| 2 | 0.01 ± 0 | 0.01 ± 0 | 0.80 ± 0.035 | 0.78 ± 0.025 |
| 4 | 0.02 ± 0 | 0.01 ± 0 | 2.73 ± 0.64 | 2.45 ± 0.49 |
| 6 | 0.01 ± 0 | 0.03 ± 0 | 1.79 ± 0.39 | 1.82 ± 0.16 |
| 8 | 0.05 ± 0 | 0.04 ± 0 | 0.09 ± 0.036 | 0.04 ± 0.034 |
| 3x-TgAD | WT | | 3xTg-AD | |
| 4 | 0.01 ± 0 | 0.01 ± 0 | 1.80 ± 0.143 | 1.75 ± 0.144 |
| 6 | 0.04 ± 0 | 0.02 ± 0 | 2.67 ± 0.46 | 3.01 ± 0.54 |
| 8 | 0.02 ± 0 | 0.02 ± 0 | 2.21 ± 0.21 | 2.22 ± 0.28 |
| 10 | 0.08 ± 0 | 0.08 ± 0 | 1.07 ± 0.14 | 1.07 ± 0.07 |

### Example 4

### In Vivo Imaging of Neutrophil Trafficking in the Brain of 5XFAD and 3xTg Mouse Models

The trafficking of neutrophils in the brain of AD mice was documented using two-photon-microscopy (TPM). Neutrophils were isolated from the bone-marrow of normal B6/SJL mice, labeled with a fluorescent dye and injected intravenously into 5XFAD and age-matched wild-type control mice. The day after, mice underwent surgical cranial procedure and acquisition under the microscope started was performed between 24-36 hours after cell injection.

Thinned Skull Preparations was used for long-term high-resolution imaging in vivo. Mice were deeply anesthetized and core body temperature was monitored and maintained using a regulated heating pad. The hair on the scalp was removed with an electric razor. The scalp was then sterilized with alcohol. An incision was made along the midline of the scalp to expose the skull overlying the cortical region of interest. Any fascia overlying the skull was scraped away with a scalpel blade. The skin and periosteum were removed. A 1 mm diameter region of skull was thinned using a high-speed micro drill and a stainless steel burr. Drilling was halted every few seconds to prevent heating and bone dust is removed using a compressed air canister. Care was taken not to deflect the skull during drilling. Drilling continued until the fine vasculature of the dura was visible. At this point, thinning continued by hand using a microsurgical blade. This process was repeated until image clarity is maximized. Animals showing any signs of damage, such as subdural or epidermal bleeding were discarded from the study. When imaging was complete, the wound margins of the scalp were sutured together using nylon suture. Mice were given a bolus of warm saline for rehydration and are allowed to recover from anesthesia on a water-circulating heating pad.

Neutrophils were isolated from bone-marrow and labeled with fluorescent cell trackers CMTPX or CMAC (Molecular probes, Life Technologies, Carlsbad, Calif., USA). Two-photon microscopy (TPM) studies were performed at 16-24 h after intravenous injection of cells. To visualize blood vessels, 20 μL of 655-nm or 525-nm non targeted Q-dots (Molecular probes, Life Technologies, Carlsbad, Calif., USA) in 100 μL of PBS were injected intravenously before mice were anaesthetized using 1.5% isoflurane with a facemask.

Time-lapse imaging was performed using a Tandem Confocal Scanning-SP5 (Leica, Germany). Each plane represents an image 525 μm by 525 μm (xy dimensions), and approximately 22-44 sequential planes were acquired at 2.5 μm increments in the z-dimension to obtain z-stacks. Z-stacks were acquired every approximately 32-63 seconds during time-lapse recordings. Image reconstruction, multidimensional rendering and manual cell tracking were done with Imaris software (Bitplane). Data were transferred and plotted in GraphPad Prism 5.0 (Sun Microsystems). The neutrophil movement analysis was performed by using functions of the T cell Analysis program (TCA; John Dempster, University of Strathclyde, Glasgow, Scotland).

We performed acquisition inside the brain parenchyma at a depth of about 150-250 μm from skull. Images were acquired from 16 hours up to 48 hours after cell injection. Notably, we observed that neutrophils adhered on vascular endothelium or migrated into the brain parenchyma at 4-4.5 months of age (Table 5). We found no evidence of neutrophil adhesion or extravasation in age-matched wild-type control mice. As demonstrated in two-photon time-lapse videos, neutrophils rolled along the endothelium, firmly adhered, crawled and extravasated into the parenchyma, where eventually displayed a high motile phenotype rapidly changing their leading and trailing edge. Importantly, neutrophils adhered and migrated in the parenchyma in the proximity of Aβ deposition.

Cell velocities are reported as the mean of instantaneous velocities in each cell track. Motility coefficients (μm²/min) were calculated for individual tracks by linear regression of displacement² versus time point with T cell analysis software. The meandering index for a cell track is computed as the displacement between the initial and final points on each the track divided by the total length of the random path.

TABLE 5
Intravascular and intraparenchymal cell movement
in AD brain

| | Intra-parenchymal | | Intra-vascular |
|---|---|---|---|
| | Directed | Non-directed | Crawling |
| Meandering | 0.73 ± 0.13 | 0.19 ± 0.01 | 0.39 ± 0.24 |
| Motility (μm²/min) | 20.84 ± 14.8 | 1.22 ± 1.59 | 3.84 ± 4.55 |
| Velocity (μm/sec) | 6.89 ± 2.78 | 3.89 ± 2.82 | 4.236 ± 1.73 |

Data are mean +/− SD

### Example 5

### Aβ₁₋₄₂ Oligomers Induce Rapid Adhesion of Neutrophils on ICAM-1 and Fibrinogen

To clarify the role of Aβ on neutrophil adhesion, we next performed in vitro rapid adhesion assays on integrin ligands. fMLP at a concentration of 0.1 or 1 μM for human or mouse cells respectively, was used as a positive control. fMLP dosage used to stimulate murine neutrophils was 10 times higher than for human neutrophils as mouse neutrophils have a second fMLP receptor subtype (FPR2), which operates at higher concentration of ligand than FPR (Hartt et al., 1999). The results demonstrate that oligomeric Aβ₁₋₄₂ induces rapid integrin-dependent rapid adhesion on fibrinogen and ICAM-1 of both human (Table 6) and mouse (Table 7) neutrophils in a dose-dependent manner. All data are expressed as the mean+/−SD of bound cells in 0.2 mm² from three independent experiments made in duplicate. The reverse Aβ₄₂₋₁ peptide had no significant effect on neutrophil adhesion. These results suggest that Aβ may activate neutrophils during early phases of disease characterized by the presence of the soluble form of Aβ, but also during later phases characterized by deposition of fibrillary peptide.

TABLE 6
Aβ₁₋₄₂ triggers rapid adhesion of human neutrophils to
Fibrinogen and ICAM-1

| | Fibrinogen | ICAM-1 |
|---|---|---|
| ctrl | 2.0 ± 1.0 | 25.8 ± 4.1 |
| fMLP (0.1 μM) | 361.5 ± 88.2 | 203.8 ± 29.2 |
| Abeta 1 μM | 108.0 ± 26.1 | 156.5 ± 40.9 |
| 5 μM | 200.9 ± 48.7 | 184.0 ± 55.4 |
| 10 μM | 285.2 ± 38.9 | 229.2 ± 43.4 |
| 20 μM | 449.1 ± 34.0 | 281.0 ± 30.7 |

TABLE 7
Aβ₁₋₄₂ triggers rapid adhesion of mouse neutrophils to fibrinogen
and ICAM-1

| | Fibrinogen | ICAM-1 |
|---|---|---|
| ctrl | 3 ± 1.0 | 15.92 ± 2.05 |
| fMLP | 72.40 ± 14.33 | 183.6 ± 23.9 |
| (1 μM) | | |
| Abeta | | |
| 1 μM | 8 ± 2.42 | 27.42 ± 3.59 |
| 5 μM | 37.13 ± 3.25 | 149.9 ± 28 |
| 10 μM | 44.75 ± 3.68 | 201.9 ± 14.14 |
| 20 μM | 88.75 ± 7.72 | 285.3 ± 12.5 |

Although two-times less efficient than the soluble form, fibrillary Aβ triggered a significant increase of rapid neutrophil adhesion on both ICAM-1 and fibrinogen (Table 8; data are expressed as the mean+/−SD of bound cells in 0.2 mm² from three independent experiments made in duplicate). These results suggest that Aβ may activate neutrophils during early phases of disease characterized by the presence of the soluble form of Aβ, but also during later phases characterized by deposition of fibrillary peptide.

TABLE 8
Both soluble and fibrillary Aβ₁₋₄₂ trigger rapid
adhesion of neutrophils to fibrinogen and ICAM-1

| | Fibrinogen | ICAM-1 |
|---|---|---|
| ctrl | 10 ± 0 | 32.5 ± 4.9 |
| fMLP | 285.5 ± 14.6 | 578.5 ± 66.5 |
| Sol Abeta 15 μM | 180.7 ± 30.7 | 480.7 ± 61.7 |
| Sol Abeta 30 μM | 419.2 ± 30.7 | 738.5 ± 29.3 |
| Fib Abeta 30 μM | 171 ± 21.8 | 457.3 ± 45.54 |

It is known that one of the receptors for Aβ is the receptor for fMLP, formylpeptide chemotactic receptor (FPR). To evaluate the contribution of this GPCR receptor on Aβ-dependent neutrophil adhesion, we inhibited its function using boc-MLF, a formyl-peptide chemotactic receptor antagonist. Human neutrophils were incubated for 2 minutes with buffer (control) or with oligomeric Aβ. 0.1 μM fMLP was used as a positive control. Values are expressed as the mean counts of bound cells in 0.2 mm² from three independent experiments in duplicate. Error bars represent SD. Table 9 shows that the adhesion of human neutrophils to ICAM-1 with both fMLP and oligomeric Aβ₁₋₄₂ was blocked by pre-treatment of cells with boc-MLF. Moreover, neutrophil adhesion on ICAM-1 was abrogated by preincubation of cells with pertussis toxin (PTx), suggesting that both fMLP and oligomeric Aβ₁₋₄₂ stimulated adhesion is mediated by Gar-coupled receptors. To exclude the possibility that rapid adhesion triggering was a consequence of a generic plasma membrane alteration due to the lipophilic nature of Aβ, we evaluated rapid adhesion in lymphocytes, which are known to lack fMLP receptors. As expected, oligomeric Aβ₁₋₄₂ did not induce adhesion to fibrinogen or to ICAM-1 in lymphocytes, further supporting the data showing FPR as a binding site for Aβ on neutrophils.

TABLE 9
Rapid neutrophil adhesion to ICAM-1 triggered by soluble
oligomeric Aβ is specifically inhibited by bocMLF and PTx.

| | bocMLF | | PTx | |
|---|---|---|---|---|
| | Ctrl | bocMLF | Ctrl | PTx |
| control | 19 ± 6.1 | 17.3 ± 6.6 | 19 ± 6.5 | 20.5 ± 8.4 |
| fMLP | 497.1 ± 33.3 | 69 ± 26.5 | 484.1 ± 39.7 | 76.4 ± 25.9 |
| Abeta | 490.4 ± 38.9 | 74.8 ± 25.5 | 475.5 ± 40.0 | 77.5 ± 19.7 |

### Example 6

### Aβ₁-42 Oligomers Trigger LFA-1 High Affinity State in Human Neutrophils

Neutrophil arrest is rapidly triggered by chemokines or other chemoattractants and is mediated by the binding of LFA-1 integrin to its vascular ligand ICAM-1. Ligation of specific heterotrimeric GPCRs by chemokines induces integrins to undergo a dramatic transition from a bent low-affinity conformation to extended intermediate- and high-affinity conformations, which leads to opening of the ligand-binding pocket. We hypothesized that Aβ may trigger intermediate and/or high affinity state of LFA-1 integrin and studied the effect of Aβ₁₋₄₂ oligomers on human LFA-1 affinity using KIM127 and 327A conformer-specific antibodies for intermediate- and high-affinity state respectively.

Neutrophils were resuspended in standard adhesion buffer at 2×10⁶/mL, and were briefly preincubated with 10 μg/ml of monoclonal antibodies KIM127 to study the extended conformation epitope corresponding to an inter-mediate-affinity state of LFA-1, or monoclonal antibody 327C to study the high-affinity state of LFA-1. The cells were stimulated for 10 s with 0.5 μmol/L of CXCL12, 0.1 μM fMLP, 20 μM Aβ1-42, or 20 μM Aβ42-1 under stirring at 37° C. After rapid washing, cells were stained with FITC-conjugated secondary polyclonal antibody and analyzed by cytofluorimetric quantification. Data are mean fluorescence intensity +/−SD from three independent experiments.

The data obtained clearly confirmed the ability of Aβ₁₋₄₂, but not scramble Aβ₁₋₄₂ peptide, to trigger LFA-1 conformation to an intermediate- and high-affinity state (Table 10), demonstrating that Aβ enhances the propensity of LFA-1 to bind its endothelial ligands and explaining the surprising capacity of neutrophils to perform arrest in areas with amyloid deposition in vivo.

TABLE 10
Aβ₁₋₄₂ triggers transition to the low-intermediate
and high affinity state of LFA-1 in human neutrophils.

| | KIM127 | 327A |
|---|---|---|
| ctrl | 1 | 1 |
| fMLP | 2.94 ± 0.21 | 4.35 ± 0.63 |
| Aβ₁₋₄₂ | 2.54 ± 0.12 | 3.29 ± 0.44 |
| Aβ₄₂₋₁ | 1.18 ± 0.17 | 1.19 ± 0.14 |

### Example 7

### Aβ₁₋₄₂ Oligomers Induce NADPH Oxidase Activation in Human Neutrophils

One of the mechanisms of neuronal damage by beta-amyloid in Alzheimer's disease is the generation of reactive oxygen species (ROS). We determined whether Aβ₁₋₄₂ oligomers induce ROS production in neutrophils by using a chemiluminescence assay.

Isoluminol-based chemiluminescence assays were performed in HGCa in the presence of horseradish peroxidase (HRP) and isoluminol (both from Sigma-Aldrich) in a temperature-controlled Multilabel Count Victor X5 (Perkin Elmer). Assays were run in dark 96 well plates precoated with 250 μg/ml human fibrinogen in HGCa containing (final concentration): 100 μM isoluminol and 8 U/ml HRP. After addition of 1×10⁵ human neutrophils or 7.5×10⁵ mouse neutrophils, plates were left at 37° C. for 5-10 min and the reaction started by addition of the stimulus. Light emission was recorded every minute.

The results shown in Tables 11 and 12 demonstrate that soluble oligomeric Aβ1-42 is able to stimulate the production of ROS in both human (Table 11) and mouse (Table 12) neutrophils plated on fibrinogen-coated well plates. We used the chemotactic peptide fMLP as positive control. The NADPH oxidase inhibitor DPI suppressed the fMLP and Aβ1-42 responses, suggesting that ROS production verified through NADPH oxidase activation (Tables 11). Data are mean+/−SD for three independent experiments.

TABLE 11
Soluble oligomeric Abeta induces ROS generation
in human neutrophils

| min | Ctrl | fMLP | Abeta | Abeta + DPI | fMLP + DPI |
|---|---|---|---|---|---|
| 1 | 73 ± 25 | 908 ± 137 | 558 ± 112 | 39 ± 1 | 40 ± 4 |
| 2 | 67 ± 27 | 783 ± 108 | 540 ± 106 | 58 ± 4 | 44 ± 3 |
| 3 | 86 ± 31 | 515 ± 103 | 358 ± 132 | 52 ± 3 | 53 ± 3 |
| 4 | 71 ± 40 | 325 ± 158 | 235 ± 61 | 55 ± 4 | 59 ± 1 |
| 5 | 87 ± 51 | 272 ± 134 | 199 ± 76 | 62 ± 2 | 66 ± 5 |

We also investigated whether the activation of NADPH oxidase could be enhanced by treatment with TNF-α up-regulates the expression and function of murine FPR in microglial cells. The results reported in Table 12 show that neutrophils treated with TNF-α presented a marked potentiation of the early phase of the production of O₂⁻ when exposed to oligomeric Aβ. Taken together these results show that ROS production induced by soluble Aβ oligomers can represent a mechanism of neutrophil-dependent injury in AD. Data are mean+/−SD counts per second.

TABLE 12
Soluble oligomeric Abeta induces ROS generation
in mouse neutrophils

| min | Ctrl | fMLP | Abeta | Abeta + TNF | TNF |
|---|---|---|---|---|---|
| 1 | 33 ± 24 | 599 ± 199 | 226 ± 34 | 2118 ± 171 | 38 ± 20 |
| 2 | 44 ± 34 | 231 ± 91 | 103 ± 17 | 947 ± 216 | 62 ± 21 |
| 3 | 47 ± 33 | 90 ± 17 | 64 ± 38 | 172 ± 66 | 102 ± 28 |
| 4 | 52 ± 34 | 65 ± 36 | 56 ± 26 | 124 ± 51 | 136 ± 26 |
| 5 | 51 ± 39 | 59 ± 54 | 60 ± 36 | 119 ± 23 | 187 ± 14 |

### Example 8

### Depletion of GR1+ Cells at the Mid-Stage of Disease Prevents Cognitive Impairment in 5XFAD and 3xTg Mice

To demonstrate a role for neutrophils in AD we depleted neutrophils or neutrophils/myeloid cells from the peripheral circulation during the early to mid-phase of disease using the highly specific anti-Ly-6G antibody (1A8 clone), which depletes neutrophils only, or anti-Gr1 mAb (RB6-8C5 clone), which recognizes Gr1 and the highly related Ly6C, which is expressed on neutrophils, dendritic cells, and subpopulations of monocytes. In vivo treatment with RB6-8C5 has been shown to deplete neutrophils and a subpopulation of GR1+Ly6C+ monocytes. Anti-small G protein Ras antibody was used as control antibody.

Briefly, the mAbs were diluted into sterile endotoxin-free PBS at a concentration of 1 mg/mL. mAbs were injected intraperitoneally at a dose of 0.5 mg per mouse for the first treatment. Then, mice were injected with 250 μg of mAbs i.p. every second day. The treatment was continued for approximately 4 weeks until behavioral testing. Neutrophil depletion was confirmed by flow-cytometric evaluation in all experimental animals. Blood neutrophil levels remained low (0-1% of blood leukocytes in treated mice, vs 8-12% in controls) when measured.

Hind limb clasping and ledge tests were first performed to assess mice motor coordination in order to determine if alterations in vestibular function might potentially cause difficulties during behavioral assessment. As expected, in both tasks, treated and not-treated 5XFAD and 3xTg mice did not perform significantly worse compared with age-matched non-transgenic control mice.

To study the impact of neutrophils on behavioral impairment, we depleted neutrophils in 5XFAD mice for 4 weeks starting at 4 months of age coinciding with the peak in neutrophil accumulation in the brain shown in Table 4 and after abnormal levels of Aβ and memory defects are known to be present. One month following the end of antibody treatment, mice were tested in the contextual fear-conditioning test (measured as percent freezing) and in the Y-maze spontaneous alternation task (percent alternation). Data are shown in Table 13.

The Y-maze spontaneous alternation task measures spatial working memory, and exploits the natural tendency of rodents to explore novel locations. Mice with intact spatial working memory are more likely to enter and explore an arm that have not recently visited rather than one that is familiar, and therefore have higher levels of spontaneous alternation. Mice making fewer than 15 total arm entries during the 8 min test were excluded from groups, in order to avoid that low numbers of entries may affect the spontaneous alternation score. The number of arms entered during the test was comparable among AD mice treated with anti-neutrophil or anti-Ras antibodies and control wild-type age-matched mice, indicating that AD mice had normal motor function and exploratory activity. As expected, a significantly decreased alternation in Y-maze task was detectable in Ras-treated (control antibody) 5XFAD mice compared to control wild-type age-matched mice. Surprisingly, treatment of 5XFAD mice with anti-Ly6G (1A8) mAb Table 13, columns A, percent alternation) or anti-Gr-1 (RB6-8C5) mab (Table 13, column B, percent alternation) almost completely blocked the cognitive deficit and mice performed at comparable levels of control wild-type healthy age-matched littermates.

TABLE 13
Depletion of neutrophils and/or neutrophil/myeloid cells restores
cognitive function in mouse models of AD

| | A: Depleting Mab = anti- | | B: Depleting Mab = anti- | |
|---|---|---|---|---|
| | LY6G (1A8 clone) | | GR-1 (RB6-8C5) | |
| Model: 5xFAD | % Freezing | % Alternation | % Freezing | % Alternation |
| WT ctrl | 62.8 ^{a} ± 3.1 | 64.7 ^{d} ± 2.4 | 61.4 ^{g} ± 3.4 | 71.1 ^{j} ± 1.9 |
| Isotype ctrl | 46.5 ^{b} ± 3.6 | 55.0 ^{e} ± 3.5 | 46.4 ^{h} ± 4.0 | 57.8 ^{k} ± 0.9 |
| PMN Depletion | 60.7 ^{c} ± 3.5 | 66.5 ^{f} ± 2.3 | 60.41 ⁱ ± 4.3 | 67.0 ^{l} ± 3.4 |

Statistical Significance:
^{a} vs ^{b}P < 0.005
^{b} vs ^{c}, ^{d} vs ^{e}, ^{e} vs ^{f}, ^{g} vs ^{h, h} vs ^{I, j} vs ^{k}P < 0.05
^{k} vs ^{l}P < 0.0005

We also performed contextual fear conditioning test, a useful tool to study hippocampus-dependent form of memory In mice, the expression of the fear memory is illustrated by freezing behavior, a lack of movement except the one necessary for breathing. In this test paradigm the associative learning of a neutral cue (sound tone) with a brief aversive stimulus (mild electric shock) is measured by monitoring the freezing behavior in mice. The fear-conditioning test was performed by placing a mouse in a box equipped with a camera for monitoring and recording the freezing behavior of the animal. The mice were first trained to associate the sound pulse with the mild electric shock, and no significant differences in freezing behavior were obvious among mice during the training period. Then, cue-dependent freezing was tested in a novel environment (one with different lighting, olfactory and visual cues) and the freezing behavior associated with the tone was measured. Wild-type control mice exhibited a robust freezing in response to the sound tone (Table 13, percent freezing), whereas, in contrast, Ras-treated-5XFAD mice were significantly impaired (Table 13, percent freezing). Treatment with Ly-6G or anti-Gr-1 antibody gave similar results leading to a drastic prevention of the memory impairment. In fact, mice treated with each of the anti-neutrophil antibody spent significantly less time freezing than age-matched 5XFAD littermates treated with the control Ras antibodies, behaving in a comparable manner to wild-type animals.

The ability of blockade of neutrophils/myeloid cells in AD pathology was explored in the 3xTg-AD model, a well-accepted model that exhibits both Aβ and tau pathology, thus more closely modeling human AD. 3x-TgAD and age-matched wild-type controls were treated with RB6-8C5 or anti-Ly6G mAb for 6 weeks starting at 6 months of age, when mice exhibit Ab deposition, synaptic dysfunction and learning and memory deficiency. After a 4 week washout/recovery period with no Mab treatment, the cognitive function of the mice was tested in both Y-maze spontaneous alternation task and contextual fear-conditioning test. The results in table 14 clearly showed that neutrophil depletion led to a dramatic reduction of cognitive deficit when compared to anti-Ras (isotype-control Mab) treatment (Table 14) returning cognitive performance to that of wild-type control animals with no disease. Data are from representative experiments with 2-14 mice per condition from a series of three with similar results.

TABLE 14
Depletion of neutrophil/myeloid cells preserves cognitive
function in mouse models of AD

| | Depleting MAb | | | |
|---|---|---|---|---|
| | Anti-Gr1 (RB6-8C5) | | Anti-LY-6G (1A8) | |
| Model: 3xTg-AD | % Freezing | % Alternation | % Freezing | % Alternation |
| WT ctrl | 39.8 ^{a} ± 2.5 | 61.6 ^{d} ± 3.6 | 46.03 ^{g} ± 2.96 | 61.62 ^{j} ± 2.36 |
| Isotype ctrl | 26.8 ^{b} ± 2.8 | 45.1 ^{e} ± 4.7 | 29.51 ^{h} ± 2.44 | 50.60 ^{k} ± 2.86 |
| Neutrophil | 41.2 ^{c} ± 3.5 | 61.8 ^{f} ± 4.8 | 45.30 ⁱ ± 6.22 | 60.15 ^{l} ± 3.46 |
| Depletion | | | | |

Statistical Significance:
^{a} vs ^{b}, ^{b} vs ^{c}, ^{d} vs ^{e}, ^{h} vs ⁱ, ^{j} vs ^{k}, ^{k} vs ^{l}P < 0.05
^{e} vs ^{f}P < 0.0005
^{g} vs ^{h}P, 0.005

Taken together these results demonstrate that therapeutic inhibition of neutrophils/myeloid cells in early to mid stage disease reverses cognition impairment and blocks cognitive decline during early/mid stage of disease.

### Example 9

### Blockade and/or Reduction of Neutrophil Interaction with Brain Reduces Neuropathological Changes Associated with AD as Measured by Normalization of Aβ, Levels, Phosphorylated Tau, Activation Status, Area and Density of Microglial Cells, and Pre- and Post-Synaptic Proteins in Brain

To understand the dramatic effect of reduction of neutrophil interaction with and/or invasion of the brain using anti-neutrophil antibodies on disease, we next studied the effect of neutrophil inhibition on Aβ, phosphorylated tau accumulation, activation status, area and density of microglial cells and several pre- and post-synaptic proteins in brains in 3xTgAD mice.

Neutrophil depletion was carried out for 4 weeks starting at 6 months of age in 3xTg-AD mice. Mice treated with the anti-neutrophil antibody anti-GR-1 showed a 98% reduction in the number neutrophils in the blood compared to those treated with the control antibody. Anti-Ras antibody was used as isotype control. Healthy C57BL/6 littermates were used as wild-type controls (WT ctrl). This treatment strategy resulted in immediate and long-term improvement in cognitive function as described in examples 8 and 10.

The relative levels of key proteins involved in AD, including Aβ, phosphorylated tau, the pre-synaptic protein synaptotagmin (encoded by gene SYT1) and PSD-95 (postsynaptic density protein 95), were measured in protein extracts from brains of isotype control and neutrophil depleted AD mice. Hemi-brains were homogenized in 1 ml TBS (120 mM NaCl, 50 mM Tris, pH 8.0) containing complete protease inhibitor (Roche Applied Science) and phosphatase inhibitor cocktail 2 (Sigma) using a Dounce homogenizer, sonicated and subsequently centrifuged at 80,000×g for 1 h at 4° C. The pellet was dissolved in 0.5 ml TBS containing 2% SDS, sonicated and centrifuged at 80,000×g for 1 h at 4° C. Supernatants (SDS-soluble fractions) and pellets were collected. Pellets (SDS-insoluble fractions) were resuspended in 0.5 ml of 70% formic acid, sonicated, centrifuged at 80,000×g for 1 h at 4° C. and supernatants were neutralized using 1 M Tris at pH 12. Equal concentrations of proteins were spotted to nitrocellulose membranes Key protein levels were measured using the following antibodies: anti-Ap Mab BAM-10; anti-total tau Mab HT7; tau phosphorylation epitopes At8 (recognizing Ser202/Thr205); and anti synaptotagmin Mab SYT1; anti-post-synaptic protein Mab PSD95; and anti-B-actin Mab. Band optical intensity was measured and quantitated; data are mean+/−SEM.

Protein quantification of Aβ1-42 levels in brain lysates revealed a dramatic reduction of Aβ1-42 levels in animals treated with anti-GR-1 antibody compared to 3xTg mice treated with anti-Ras control antibody (Table 15). Both soluble fraction and SDS-extracted supernatant rich in pre-amyloid species were observed. Surprisingly, we observed that neutrophil depletion reversed amyloid deposition to the level of healthy control mice. For Tau protein quantification we used the same samples used for Aβ quantification. Western blot of phosphorylated tau at Ser202/Thr205 (using MAb AT8) revealed an increase in tau phosphorylated in the soluble fraction in animals treated with anti-Ras antibody and a marked reduction of phospho-tau in animals treated with anti-Gr-1 antibody (Table 15). These results demonstrate a role for neutrophils in amyloid deposition and induction of tau phosphorylation, two pivotal mechanisms in the induction of behavioral impairment in AD-like disease, and suggest that inhibition of neutrophil function may represent a new therapeutic approach in AD.

TABLE 15
Neutrophil/myeloid cell depletion results in normalization of Abeta and
phosphorylated tau in mouse model of AD

| Model: 3xTg-AD | Abeta | Phosphorylated Tau | B-actin |
|---|---|---|---|
| isotype ctrl | 2.41 ± 2.44 | 1.75 ± 0.26 | 1.40 ± 0.21 |
| anti-Gr1 | 1.04 ± 0.11 | 0.67 ± 0.17 | 1.35 ± 0.17 |

Synaptic dysfunction is considered a significant factor contributing to memory loss in Alzheimer's disease, correlates better with cognitive dysfunction than amyloid deposition and tangle formation, and is recognized to be a primary pathological target for treatment. Reduced levels of the pre-synaptic protein synaptotagmin are documented in the brain and CSF of patients with AD, including subjects at an early stage of disease. The levels of synaptotagmin are reduced in the AD mice treated with the isotype control Mab compared to the WT, age-matched control animals (Table 16). Reduction of neutrophil/myeloid cells results in normalization of synaptotagmin levels (Table 16.). The post-synaptic protein PSD95 is also reduced in AD patients and in models of AD; however at very late timepoints (12+ months). At the mid-stage of disease there was trend to reduced levels of PSD95 in the 3xTG-AD model was observed (Table 16) in the isotype-control Mab treated AD mice compared to the wild-type controls which was normalized by depletion by neutrophil/myeloid cells.

TABLE 16
Neutrophil/myeloid cell depletion in mouse model
of AD results in normalization of pre-synaptic protein
synaptotagmin and post-synaptic protein PSD95.

| | PSD95 | Synaptotagmin |
|---|---|---|
| WT ctrl | 1.54 ± 0.25 | 1.10^{a} ± 0.20 |
| Isotype ctrl | 1.48 ± 0.14 | 0.67^{b} ± 0.07 |
| Anti-Gr1 | 1.64 ± 0.19 | 1.14^{c} ± 0.12 |

^{a} vs ^{b}P < 0.05
^{c} vs ^{d}P < 0.05

Microglia are the resident macrophages of the brain and spinal cord and can be identified by various markers including Iba-1. Histological evaluation of the microglial cells in brain, using Iba-1+ as a marker of microglial, cells, revealed a highly activated phenotype of microglial cells, defined as cells with an enlarged cell body and thick, retracted processes, in the control-antibody treated AD mice as compared to unactivated microglial cells, defined as cells with small, round soma and long processes, in neutrophil/myeloid depleted mice.

The numerical density of Iba-1⁺ immunoreactive microglia was determined in 4 non-consecutive coronal sections throughout the cortex and the dorsal hippocampus of both 3xTg-AD and WT control mice. The specific analyzed areas were the parietal cortex, the dentate gyrus and the CA1 hippocampus. Iba-1⁺ microglia cells were visualized using a LEICA fluorescence microscope (DM6000B, Leica) and counted blindly with ImageJ 1.32j software. Unbiased quantitative stereologic analysis was performed on cortex slices to determine the total number and area of Iba-1+ cells (Table 17). Microglia cell density and area was lower in neutrophil-depleted animals and the microglial cells displayed a non-activated phenotype, In neutrophil-depleted AD mice compared to isotype-control treated AD mice (Table 17).

TABLE 17
Blockade of neutrophil interaction with
the brain reduces the number and
area of microglial cells in the brain.

| | Density | Area |
|---|---|---|
| Isotype ctrl | 48.83^{a} ± 3.09 | 538.4^{c} ± 23.74 |
| Anti-Gr1 | 35.83^{b} ± 1.08 | 441.7^{d} ± 34.14 |

^{a} vs ^{b}P < 0.005
^{c} vs ^{d}P < 0.05

Taken together these results demonstrate that therapeutic inhibition of neutrophils/myeloid cells in early to mid stage disease reduces and/or reverses neuropathological changes that occur in AD and thus represents a therapeutic approach to treat and/or prevent AD.

### Example 10

### Neutrophil Blockade has Long-Term Protective Effect on Cognitive Functions

We demonstrated a long-term effect of neutrophil blockade during early disease. Short-term reduction of neutrophil interaction with and/or invasion of the brain was demonstrated using short-term depletion of neutrophils. 3x-TgAD and age-matched wild-type controls were treated with the anti-GR-1 Mab RB6-8C5 for 6 weeks starting at 6 months of age, when mice exhibit Ab deposition, synaptic dysfunction and learning and memory deficiency. 3xTg mice of 6 months old were treated in the first day with 500 μg anti-Gr1 antibody i.p. and then the treatment continued with 250 μg of antibody every other day for 6 weeks. Mice were left untreated for 6 months. Following 6 months of no treatment, cognitive performance was tested in the contextual fear-conditioning test. Short-term neutrophil depletion led to a dramatic, sustained, long-term reduction of cognitive deficit when compared to isotype matched control (anti-Ras) treatment. Surprisingly, cognitive behavior was the same as healthy, wild-type control animals (Table 18). We found that the restoration of cognitive function to wild-type condition in anti-Gr-1 treated mice was completely maintained also at later time points of disease, as shown by the results obtained from the contextual fear-conditioning test (Table 18). Moreover, control animals treated with anti-Ras antibody had a clear tendency of being immobile in Y maze test, whereas, in contrast, anti-Gr1-treated mice had a significantly higher tendency of moving and exploring normally the maze, suggesting a neuroprotective effect of treatment with anti-Gr1 antibody (Table 18).

TABLE 18
Short-term blockade of neutrophil adhesion and migration
into the brain using neutrophil depletion during early-
mid stage disease provides long-term preservation of
cognitive function in aged mouse model of AD

| Model: 3xTg-AD | % Freezing |
|---|---|
| WT ctrl | 41.78^{a} ± 6.13 |
| Isotype ctrl | 6.02^{b} ± 1.12 |
| Neutrophil depletion (Anti-Gr1) | 37.65^{c} ± 5.55 |

Statistical significance:
^{a} vs ^{b}P < 0.05
^{b} vs ^{c}P < 0.05

These results show that brief therapeutic intervention at mid-stage disease aimed to block neutrophil trafficking/function and interaction with the brain inhibits/reverses cognitive deficits, blocks cognitive decline, and has beneficial long-term effect on disease.

### Example 11

### Short-Term Blockade of Neutrophil Adhesion and Migration into the Brain During Early-Mid Stage Disease Provides Medium and Long-Term Preservation of Cognitive Function in Aged 3xTG AD Mice

To further support these results we also evaluated the long-term effect of short-term, mid-stage inhibition of neutrophil function, interaction with the brain and trafficking using anti-adhesion molecule therapy. Neutrophils express high levels of the β2 integrins CD11a/CD18 (LFA-1) and CD11b/CD18 (Mac-1). As CD11a/CD18 was shown to be fundamental in the control of firm adhesion on vascular endothelium, we asked whether blockade of LFA-1-mediated adhesion interferes with the migration of neutrophils in the brain parenchyma and consequently with AD pathology. We treated 3xTg mice of 6 months of age with a single dose of 500 μg of LFA-1 integrin-specific mAb (TIB213 clone) followed by treatment with 300 μg of antibody every other day for 6 weeks. Following a 4 week followed by 6 months of no-treatment. 6 months after the termination of the anti-LFA-1 washout and recovery period animals were tested in in the spontaneous alternation task and the contextual fear-conditioning test (Table 19). treatment, mice were tested for behavioral assessment with Y-maze spontaneous alternation task and contextual fear-conditioning test. As shown for anti-Gr-1 treatment, blockade of the integrin LFA-1 at early stages of AD-like pathology had long-term effect and preserved the cognitive impairment also at later time points of disease, as it is shown by contextual fear-conditioning test (Table 19). Importantly, the treatment with anti-LFA-1 antibodies at early time-points of disease allowed the re-establishment of the cognitive function almost to wild-type condition in aged AD mice (Table 19). Moreover, we observed a significant tendency of control anti-Ras-treated AD mice of being immobile in the maze during the Y maze test, whereas, in contrast, anti-LFA-1-treated mice had a greater tendency of exploring the maze normally, (Table 19).

TABLE 19
Short-term blockade of neutrophil adhesion and migration into the
brain using anti-adhesion molecule Mab provides long-term
preservation of cognitive function in aged mouse model of AD

| | | | Late-stage |
|---|---|---|---|
| | Mid-stage Testing | | Testing |
| Model: | (8 mo) | | (13 mo) |
| 3xTg-AD | % Freezing | % Alternation | % Freezing |
| WT ctrl | 41.94^{a} ± 2.17 | 69.74^{d} ± 0.83 | 48.3^{g} ± 4.62 |
| Isotype ctrl | 28.06^{b} ± 3.96 | 45.69^{e} ± 2.37 | 20.43^{h} ± 3.42 |
| Anti-LFA-1 | 45.51^{c} ± 1.99 | 56.53^{f} ± 1.67 | 37.49ⁱ ± 3.85 |

Statistical significance:
^{a} vs ^{b}, ^{d} vs ^{e}P < 0.005
^{b} vs ^{c}, ^{e} vs ^{f}, ^{g} vs ^{h}P < 0.005
^{h} vs ^{l}P < 0.05

Short-term blockade of neutrophil/myeloid cell function and interaction with the brain using anti-LFA-1 therapy (for 4 weeks starting at 6 months) followed by a 6 month period of no treatment and retesting at month 13 revealed long-term reduction in the number and activation status of microglial cells in both the CA1 and dentate gyrus regions of the hippocampus (Table 20)

TABLE 20
Short-term blockade of neutrophil/myeloid cell function and interaction with the brain using
antii-LFA-1 therapy provides long-term reduction in microglial activation in the hippocampus.

| | | | Microglia in | |
|---|---|---|---|---|
| Model: | Microglia in the CA1 | | the dentate gyrus | |
| 3xTg-AD | Density CA1 | Area CA1 | Density DG | Area DG |
| Control treatment | 64.75^{a} ± 6.99 | 431.3^{c} ± 11.46 | 87.50^{e} ± 2.9 | 420.8^{g} ± 5.54 |
| Anti-LFA1 | 31.75^{b} ± 1.18 | 315.0^{d} ± 6.45 | 30^{f} ± 1.08 | 312.9^{h} ± 13.24 |
| treatment | | | | |

### Example 12

### Confirmation of the Role of LFA-1 in AD

The important role of LFA-1 integrin was confirmed by crossing the 3xTg-AD animals with the LFA-1 deficient Itgal−/− strain to produce AD mice deficient in LFA-1 (KOLFA-1)/3xTg-AD). At 9 months of age these mice showed normal motor functions and typical exploratory behavior in the Y-maze spontaneous alteration task comparable to wild-type age-matched littermates (Table 21).

TABLE 21
AD mouse model 3xTg-AD lacking LFA-1
integrin display normal cognitive behavior

| | % Alternation |
|---|---|
| WT ctrl | 57.36^{a} ± 1.63 |
| 3xTg-AD | 50.97^{b} ± 2.37 |
| KOLFA-1/3xTg-AD | 59.38^{c} ± 2.68 |

^{a} vs ^{b}, ^{b} vs ^{c}P < 0.05

TABLE 22
LFA-1-deficient 3x-TgAD mouse model of AD have fewer, less activated microglia in both
the CA1 and dentate gyrus areas of the hippocampus compared to 3x-TgAD age-matched controls

| | Microglia in CA-1 | | Microglia in dentate gyrus | |
|---|---|---|---|---|
| | Density | Area | Density | Area |
| 3xTg-AD | 34.11^{a} ± 3.5 | 403.4^{c} ± 10.95 | 34.75^{e} ± 2.3 | 369.2^{g} ± 7.69 |
| KOLFA-1/3xTg-AD | 20.50^{b} ± 2.54 | 347.4^{d} ± 6.88 | 26.89^{f} ± 1.68 | 324.5^{h} ± 7.2 |

^{a} vs ^{b}, ^{c} vs ^{f}P < 0.05
^{c} vs ^{d}, ^{g} vs ^{h}P < 0.005

This example confirms a key role of LFA-1 integrin in the pathogenesis of AD and indicates LFA-1 as a target for prevention and treatment of AD.

### Example 13

### Treatment with Anti-Alpha4 Integrin at the Mid-Stage of Disease Prevents Cognitive Impairment in 3xTg Mice

We demonstrated a long-term effect of neutrophil blockade during early disease. 3xTg mice at 6 months old were treated in the first day with 500 μg anti-alpha4 antibody (clone PS/2) i.p. and then the treatment continued with 250 μg of antibody every other day for 6 weeks. Then mice were tested in a behavioral paradigm after the termination of treatment. We found a dramatic restoration of cognitive function almost to wild-type condition in anti-alpha4 treated mice as shown by the results obtained from the contextual fear-conditioning test (Table 23). These experiments clearly show that alpha4 integrins are key molecular mechanism in neutrophil-mediated behavioral impairment in AD-like disease and indicate that, as shown for anti-LFA-1 treatment, brief therapeutic intervention with anti-alpha 4 antibody at mid-stage disease may inhibit cognitive deficits and have beneficial long-term effect on disease.

TABLE 23
Blockade of neutrophil/myeloid function with
anti-alpha-4 integrin Mab at the early to mid-
stage of disease provides long-term prevention
of cognitive impairment in a mouse model of AD

| Model: 3xTg-AD | % Freezing |
|---|---|
| WT ctrl | 48.4^{a} ± 3.38 |
| Isotype ctrl | 32.9^{b} ± 3.93 |
| Anti-alpha4 | 46.3^{c} ± 4.25 |

Statistical significance
^{a} vs ^{b}P < 0.005
^{c} vs ^{d}P < 0.05

### Example 14

### Neutrophils Invade Human AD Brains and Form NETs

We evaluated human cortical and hippocampal brain tissue from subjects with AD (12 sporadic AD patients) compared with 2 Down syndrome patients, 8 age-matched control patients and 3 healthy controls.

Brain sections from CNS autopsy tissues were embedded in paraffin and processed for histopathological and histochemical analyses. Hippocampal and cortical sections were used for double immunostaining and labeled with polyclonal rabbit anti-human myeloperoxidase (1:300, Dako) and anti-human beta-amyloid (1:50, clone 6F/3D Dako). Epitope retrieval was obtained by heat-induction with 0.1 M citrate buffer at pH 6.0. For anti-human beta-amyloid staining, tissue was pre-treated with 100% formic acid (Carlo Erba reagents). Subsequently, sections were treated with 20% normal goat serum (Vector) and 1% BSA and then incubated with primary antibodies overnight at 4° C. After washing with PBS, appropriate biotinylated secondary antibody (Texas Red, Vector), and fluorophore-conjugated secondary antibody (anti-rabbit-Alexa488, Molecular Probes, Invitrogen) were added. Nuclei were stained with DAPI (Abbott Molecular Inc.).

MPO positive cell quantification: for the automatic quantification, human brain slices were acquired with Tandem Confocal Scanning-SP5 at magnification 10× with a resolution of 1024×1024 pixels (1551×1551 μm of area). 10 random acquisitions were performed for each section with a median z-volume of 37 μm. Before cell quantification, image acquisitions were converted in TIFF image format. The cell counting method consisted of six phases: rolling ball for background subtraction, color deconvolution, brightness threshold for acquiring a binary image, Gaussian blur for smoothening of the threshold, watershed segmentation of cells touching each other and analyze particles tool for finding out the cell count. The commands were recorded in as a macro for ImageJ enabling a continuous, automated analysis of the images by the program.

We demonstrated elevated neutrophil numbers in AD patients compared to control age-matched subjects, suggesting a role for neutrophils in humans as well (Table 18). Images in AD patients revealed spread/adhered neutrophils in cortical brain vessels, displaying a distinct front or leading edge characteristic of cell polarization before migration. As shown above in animal models of AD, we found proximity between amyloid plaques/angiopathy and neutrophils, suggesting a role for amyloid deposition in neutrophil infiltration. The quantification of MPO positive cells in AD patient brain tissues clearly showed a significant higher number of neutrophils compared to age-matched subjects both inside blood vessels and in the parenchyma (Table 18). Of note, most neutrophils in AD patients were apparently migrated inside brain parenchyma, whereas in contrast, the low number of neutrophils found in age-matched subjects was mostly confined in blood vessels. Furthermore, we found that neutrophils produced NETs inside blood vessels and inside the parenchyma, confirmed by the co-localization of MPO staining and citrullinated histones and of MPO and neutrophil elastase. In support of the results found in human AD brains, we found that neutrophils release NETs also in the brain of 5XFAD mice suggesting that NETosis may represent a novel disease mechanism in the pathogenesis of AD.

TABLE 24
Elevated numbers and distribution of
neutrophils in brain of AD patients
compared to aged-matched control subjects

| | Inside | |
|---|---|---|
| | Blood | Inside |
| | Vessel | Parenchyma |
| Aged-matched Ctrl | 1.72^{a} ± 0.30 | 0.49^{c} ± 0.18 |
| AD Patients | 4.53^{b} ± 0.64 | 9.71^{d} ± 1.23 |

Statistical significance
^{a} vs ^{b}P < 0.005
^{c} vs ^{d}P < 0.0005

### Example 15

### Blockade of Protein Tyrosine Kinase (PTK) Signaling Activity Using PTK Inhibitors, Including Multiple Inhibitors to JAK, ABL1, BTK and SYK, are Able to Prevent Neutrophil Adhesion Triggered by Aβ

Since Aβ is able to induce neutrophil adhesion, and neutrophils found in both mouse and human AD brains are largely localized to areas of Aβ deposition, Aβ-induced neutrophil adhesion and extravasation is a key target for treatment of AD. As a model system, human primary PMNs were isolated form healthy donors stimulated to very rapid adhesion to purified human ICAM-1, ligand for the β2 integrins LFA-1 and Maci. fMLP (the classical chemoattractant) was the positive control. Standard adhesion assays were performed under static conditions (although APP also triggers arrest under flow). PMNs were treated for 30 min. with different concentrations of inhibitors followed by stimulation for 1 min with 50 nM fMLP or with 10 μM Aβ. The number of adherent cells was quantified by computer-assisted evaluation. Statistical analysis was SD.

Aβ and fMLP were equally potent agonists of integrin-mediated rapid adhesion to ICAM-1 (Table 25). Both act through FPR1, as shown by the capability of the FPR1 inhibitor BOC-MLP to block triggered adhesion by fMLP and Aβ. Pertussis toxin blocks adhesion triggered either by fMLP or Aβ confirming the involvement of G-protein coupled receptors. JAK, ABL1, BTK and SYK inhibitors are able to prevent neutrophil adhesion triggered by fMLP and Ap (Table 25).

Thus, blockade of molecules in signaling pathways involved in Aβ-triggered neutrophil activation and adhesion is a useful approach for treatment of AD. Further, blockade of protein tyrosine kinases, including but not limited to JAK, ABL1, BTK and SYK is a pharmacological strategy to prevent PMN interaction with the vasculature and/or recruitment to the brain triggered by Aβ signaling during Alzheimer's disease.

TABLE 25

| | | | Adherent hPMNs to ICAM-1 | | | | | |
|---|---|---|---|---|---|---|---|---|
| | | | n.t. (resting) | | fMLP | | Aβ oligo | |
| Targets | Inhibitors | | Mean | SD | Mean | SD | Mean | SD |
| Galphai | Control | | 28.0 | 6.9 | 484.1 | 39.7 | 475.5 | 39.0 |
| | PTx 2 | μg/ml | 27.9 | 6.4 | 76.4 | 25.9 | 77.5 | 19.7 |
| FPR1 | Control | | 25.9 | 9.6 | 486.8 | 35.7 | 470.7 | 45.7 |
| | BOC-MLP 100 | μM | 20.1 | 6.6 | 55.0 | 24.6 | 81.8 | 26.1 |
| JAKs | Control | | 24.8 | 7.6 | 444.6 | 73.8 | 464.0 | 73.5 |
| | AG490 200 | μM | 23.2 | 7.7 | 274.5 | 82.7 | 258.6 | 54.5 |
| | WHI-P154 100 | μM | 23.5 | 7.0 | 207.8 | 51.4 | 218.8 | 57.7 |
| | P1-TKIP 40 | μM | 22.9 | 7.8 | 314.6 | 55.4 | 430.5 | 86.3 |
| | CP-690550 200 | μM | 22.1 | 6.4 | 209.6 | 57.3 | 172.4 | 75.3 |
| | Ruxolitinib 100 | μM | 17.0 | 5.7 | 243.8 | 67.7 | 244.5 | 65.8 |
| | TG101348 100 | μM | 20.1 | 6.0 | 147.2 | 51.4 | 134.8 | 53.9 |
| SYK | Control | | 23.4 | 6.6 | 500.8 | 53.0 | 500.8 | 38.7 |
| | Piceatannol 50 | μM | 21.0 | 5.9 | 218.7 | 70.3 | 276.1 | 52.3 |
| | PRT 2.5 | μM | 21.3 | 6.6 | 197.3 | 65.4 | 242.6 | 63.4 |
| ABL1 | Control | | 23.7 | 7.6 | 433.5 | 71.1 | 464.5 | 64.0 |
| | Imatinib 100 | μM | 23.5 | 6.6 | 124.9 | 42.4 | 109.3 | 30.8 |
| | Dasatinib 20 | μM | 24.9 | 7.1 | 230.8 | 55.7 | 227.5 | 73.5 |
| | Nilotinib 200 | μM | 24.3 | 6.7 | 245.5 | 56.4 | 265.6 | 85.5 |
| | GNF-2 100 | μM | 25.2 | 8.0 | 144.2 | 76.9 | 115.5 | 30.8 |
| | Bosutinib 100 | μM | 19.3 | 5.8 | 98.2 | 25.2 | 95.1 | 26.5 |
| BTK | Control | | 24.2 | 7.4 | 463.1 | 61.8 | 455.0 | 65.3 |
| | Ibrutinib 100 | μM | 21.9 | 6.4 | 80.0 | 30.9 | 78.0 | 27.2 |

What is claimed is:

1. A method of prevention and treatment of neurodegenerative disease in an individual mammal, said method comprising:
administering to said individual mammal an effective amount of an agent that reduces the presence or activity of myeloid cells and/or neutrophils in the brain.

2. The method of claim 1, wherein the neurodegenerative disease is Alzheimer's disease.

3. The method of claim 2, wherein the treatment reduces development of cognitive deficits in the mammal.

4. The method of claim 3, wherein the individual is diagnosed with AD prior to treatment.

5. The method of claim 4, wherein the individual is differentially diagnosed with AD.

6. The method of claim 1, wherein the mammal is a rodent that provides a model for AD.

7. The method of claim 1, wherein the mammal is a human.

8. The method of claim 1, wherein the agent has an activity selected from: (i) depletion of neutrophil/myeloid cell populations systemically or locally in the brain; (ii) blocking neutrophils/myeloid cell adhesion and crawling; (iii) blocking transmigration and infiltration of neutrophils/myeloid cells into the brain; (iv) blocking cell-cell interactions between neutrophil/myeloid cells and endothelial cells and/or neural cells; (v) blocking neutrophil/myeloid cell extracellular-matrix interactions; (vi) reducing motility of neutrophils/myeloid cells in the brain parenchyma; (vii) blocking Aβ-induced activation and adhesion of neutrophils/myeloid cells; (viii) blocking intracellular signaling controlling adhesion and activation; (ix) blocking neutrophil activation and/or degranulation; (x) blocking release of reactive oxygen species, proteases, cytokines, lipid mediators or other damaging agents from myeloid cells and/or neutrophils; (xi) blocking neutrophil/myeloid cell activation leading to increased affinity and valency; (xii) blocking formation of neutrophil extracellular traps (NETS) in brain vessels or parenchyma; (xiii) blocking neurodegenerative processes including synaptic dysfunction and/or degradation; (xiv) reducing activation and/or number of microglial cells.

9. The method of claim 8, wherein the agent inhibits the interaction between an adhesion molecule involved in leukocyte trafficking or extravasation and a ligand for the adhesion molecule.

10. The method of claim 9, wherein the adhesion molecule is selected from ICAM-1, LFA-1, CD11a, CD11b, CD11c, CD18, alpha-4 integrin, E-selectin, P-selectin and L-selectin.

11. The method of claim 9, wherein the ligand is selected from VCAM-1, MAdCAM-1, CD49; PSGL-1, CD44, CD43, and hyaluronan.

12. The method of claim 8, wherein the agent depletes neutrophil/myeloid cell populations systemically, or locally in the brain.

13. The method of claim 8, wherein the agent inhibits activity of a protein tyrosine kinase involved in leukocyte activation or trafficking.

14. The method of claim 13, wherein the protein tyrosine kinase is selected from Syk, Abl, JAK3, Jak2, and BTK and MAPK; and PI3K.

15. The method of any one of claim 1-14, wherein the agent does not cross the blood brain barrier after administration.

16. The method of claim 1, wherein the efficacy of treatment is tracking by monitoring one or more biomarkers selected from (i) the number of circulating neutrophils/myeloid cells and/or ratio of circulating neutrophils/myeloid cells and other leukocytes; (ii) the number of brain-resident neutrophils/myeloid cells; (iii) activation status of circulating neutrophils/myeloid cells; (iv) activation status of brain-resident neutrophils/myeloid cells; (v) adhesion capability of circulating neutrophils/myeloid cells; (vi) adhesion capability of brain-resident neutrophils/myeloid cells; (vii) inflammatory markers in blood; (viii) inflammatory markers in cerebrospinal fluid; (ix) neurodegenerative markers in cerebrospinal fluid.

17. The method of claim 16, wherein monitoring is performed at multiple time points.

18. A kit for use in the methods of any one of claims 1-17, comprising an agent and instructions for use.

19. A unit dose of a medicament for us in the methods of any one of claims 1-17.
